(19) United States
(12) Patent Application Publication
(10) Pub. No.: US 2010/0272660 A1
(43) Pub. Date: Oct. 28, 2010

(54) COSMETIC OR PHARMACEUTICAL COMPOSITION COMPRISING A POLYCONDENSATE, THE SAID POLYCONDENSATE AND METHOD OF COSMETIC TREATMENT

(76) Inventor: Gerard Malle, Villiers s/Morin (FR)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/681,473

(22) PCT Filed: Oct. 2, 2008
(86) PCT No.: PCT/FR08/51782

§ 371 Date: Jun. 14, 2010

Related U.S. Application Data
(60) Provisional application No. 60/984,739, filed on Nov. 2, 2007.

(30) Foreign Application Priority Data
Oct. 4, 2007 (FR) 0758057

(51) Int. Cl. A61K 8/89; A61Q 1/10; A61K 31/695; C07F 7/08; A61P 43/00; A61Q 1/04; A61Q 3/00; A61Q 13/00; A61Q 5/00; A61Q 17/04; A61Q 19/00; A61Q 5/10

(52) U.S. Cl. 424/59; 424/61; 424/63; 424/64; 424/70.1; 424/70.6; 424/70.7; 514/63; 556/437; 8/405

(57) ABSTRACT

The present application relates to a cosmetic or pharmaceutical composition comprising a polycondensate capable of being obtained by the reaction of the following monomers alone:
of 10 to 30% by weight, relative to the total weight of the polycondensate, of one or more polyols comprising 3 to 6 hydroxyl groups;
of 30 to 80% by weight, relative to the total weight of the polycondensate, of one or more linear, branched and/or cyclic, saturated or unsaturated, non-aromatic monocarboxylic acids comprising 6 to 32 carbon atoms;
of 1 to 40% by weight, relative to the total weight of the polycondensate, of one or more polycarboxylic acids and/or cyclic anhydrides of such a polycarboxylic acid and/or lactones comprising at least one COOH group;
   and optionally of 0.1 to 15% by weight, relative to the total weight of the polycondensate, of one or more silicones having a hydroxyl and/or carboxyl functional group.

The application also relates to a method of cosmetic treatment using the said composition, and the polycondensate thus defined.

The present invention relates to cosmetic compositions, in particular make-up and especially lipsticks, comprising particular polycondensates.

Numerous cosmetic compositions exist for which gloss properties of the film deposited, after application to keratin materials (skin, lips, superficial body growths), are desired. There may be mentioned, for example, lipsticks, nail varnishes or certain hair products.

In order to obtain such a result, it is possible to combine specific raw materials, in particular lanolins, with so-called glossy oils, such as polybutenes which have nevertheless a high viscosity; or esters of a fatty acid or a fatty alcohol in which the number of carbons is high; or alternatively certain vegetable oils; or else esters resulting from the partial or complete esterification of a hydroxylated aliphatic compound with an aromatic acid, as described in patent application EP 1097699.

It is also known to combine lanolins with polyesters obtained by sequential reaction of castor oil with isostearic acid and then with succinic acid, as described in patent U.S. Pat. No. 6,342,527.

To improve the gloss of the deposited film, and its staying power, it has also been proposed to use esters resulting from the condensation of a polyol with a “new” type carboxylic acid, in particular in FR 2838049.

There may also be mentioned EP 1457201, which describes a composition combining a polyester of triglycerides of hydroxylated carboxylic acids and an oil of low molecular mass chosen from polybutylenes, hydrogenated polyisobutylenes, hydrogenated or non-hydrogenated polydecenes, copolymers of vinylpyrrolidones, esters of linear fatty acids, hydroxylated esters, esters of branched C24-C28 fatty alcohols or fatty acids, silicone oils and/or oils of plant origin.

In patent application EP 0792637, a composition combining an aromatic ester or a polymer of the polybutene or polyisobutene type is described.

In patent application EP 1155687, a process is described which consists in incorporating, into an oily phase constituted of a cosmetically acceptable oil, an organopolysiloxane having at least 2 groups capable of establishing hydrogen bonds.

However, these compositions and combinations, even though they significantly improve the gloss, are still judged inadequate for the purpose of a long staying power of this gloss, over time.

The aim of the present invention is to provide novel polymers which may confer a significant gloss on a deposit, in particular a film-forming deposit, while maintaining good stability of this gloss over time; it may find a particularly advantageous application in the field of lipsticks. Moreover, polymers are also sought which can additionally advantageously confer on the composition excellent staying power over time on keratin materials, in particular on the lips.

For this purpose, the applicant has sought novel alkyd-type polycondensates having the desired properties.

Alkyd resins constitute a particular class of polyesters in being the product of reaction of polyols and polycarboxylic acids, generally modified by unsaturated fatty acids, such as oleic acid, or by unsaturated oils, for example soybean or castor oil, which make it possible to modulate their film-forming properties, in particular their rate of drying, their hardness and their resistance.

Thus, there has been proposed in the document U.S. Pat. No. 2,915,488 modified alkyd resins in which part of the fatty acids derived from soybean oil has been replaced by benzoic acid. These novel resins have improved properties in terms of resistance to alkalis and to detergents; the films containing them dry more rapidly and are harder. However, no application, in particular no cosmetic or topical application, has been envisaged for these resins. Furthermore, the composition of the alkyd resins of this document are not optimal, for use in cosmetics, in particular in terms of stability of the polymers.

WO 2006/004911 is also known which describes polyol polyester polymers which can be used in cosmetics, in particular for replacing castor oil; however, all the polymers described in this document have a dynamic viscosity at 25° C. of between 200 and 5000 centipoises (mPa·s). Nevertheless, these polymers do not confer on the compositions containing them the desired properties in terms of gloss, maintaining this gloss over time, and of long staying power of the composition on the keratin materials, the skin in particular.

Moreover, most known alkyd resins do not have suitable solubility in the oily media normally used in cosmetics, such as vegetable oils, alkanes, fatty esters, fatty alcohols, silicone oils, and in particular comprising isododecane, Parleam, isononyl isononanoate, octyldodecanol, phenyltrimethicone, C12-C15 alkyl benzoate and/or D5 (decamethylcyclopentasiloxane).

After major research studies, the applicant has discovered surprisingly and unexpectedly that certain polycondensates with a high content of particular carboxylic acids, and with a very high viscosity, could lead to improved performance in terms of gloss, maintenance of the said gloss, and additionally long staying power of the film obtained, while being capable of being carried in the customary cosmetic media, in particular the customary cosmetic oily media.

The subject of the present invention is therefore a cosmetic or pharmaceutical composition comprising, in a cosmetically or pharmaceutically acceptable medium, at least one polycondensate capable of being obtained by the reaction of the following monomers alone:
of 10 to 30% by weight, relative to the total weight of the polycondensate, of one or more polyols comprising 3 to 6 hydroxyl groups;
of 30 to 80% by weight, relative to the total weight of the polycondensate, of one or more linear, branched and/or cyclic, saturated or unsaturated, non-aromatic monocarboxylic acids comprising 6 to 32 carbon atoms;
of 1 to 40% by weight, relative to the total weight of the polycondensate, of one or more polycarboxylic acids and/or cyclic anhydrides of such a polycarboxylic acid and/or lactones comprising at least one COOH group;
   and optionally of 0.1 to 15% by weight, relative to the total weight of the polycondensate, of one or more silicones having a hydroxyl and/or carboxyl functional group.

The subject of the invention is also the polycondensate thus defined.

In particular, the cosmetic compositions exhibit good applicability and good covering power; good adhesion to the support, whether on the nail, the hair, the eyelashes, the skin or the lips; adequate flexibility and resistance of the film, so as to avoid cracks, for example in the case of lipsticks or varnish; and also an excellent level of long-lasting gloss. The comfort and slip properties are also very satisfactory. These polycondensates can be easily carried in cosmetic solvent or oily media, in particular oils, fatty alcohols and/or fatty esters, which facilitates their use in the cosmetic field, in particular in lipsticks or foundations.

The polycondensates according to the invention may be easily prepared, in a single synthesis step, and without producing waste, at low cost.

Moreover, it is possible to easily modify the structure and/or the properties of the polycondensates according to the invention by varying the chemical nature of the various constituents and/or their proportions.

The polycondensates according to the invention are advantageously branched; it is possible to think that this makes it possible to generate a network by entanglement of the polymer chains, and to therefore obtain the desired properties, in particular in terms of improved staying power, improved gloss, and in terms of solubility. It has indeed been observed that linear polycondensates do not make it possible to obtain a notable improvement in the staying power of the composition, and that polycondensates of the dendrimer type, whose chains are regular, do not have optimum solubility.

The polycondensates according to the invention are polycondensates of the alkyd type, and are therefore capable of being obtained by esterification/polycondensation, according to methods known to a person skilled in the art, of the constituents described below.

One of the constituents necessary for the preparation of the polycondensates according to the invention is a compound comprising 3 to 6 hydroxyl (polyol) groups, in particular 3 to 4 hydroxyl groups. It is quite obviously possible to use a mixture of such polyols.

The said polyol may in particular be a saturated or unsaturated, linear, branched and/or cyclic carbon-containing, in particular hydrocarbon-containing, compound comprising 3 to 18 carbon atoms, in particular 3 to 12, even 4 to 10 carbon atoms, and 3 to 6 hydroxyl (OH) groups, and capable of additionally comprising one or more oxygen atoms intercalated in the chain (ether functional group).

The said polyol is preferably a linear or branched, saturated hydrocarbon compound comprising 3 to 18 carbon atoms, in particular 3 to 12, even 4 to 10 carbon atoms, and 3 to 6 hydroxyl (OH) groups.

It may be chosen from, alone or as a mixture:
triols, such as 1,2,4-butanetriol, 1,2,6-hexanetriol, trimethylolethane, trimethylolpropane, glycerol;
tetraols, such as pentaerythritol (tetramethylolmethane), erythritol, diglycerol or ditrimethylolpropane;
pentols such as xylitol;
hexols such as sorbitol and mannitol; or alternatively dipentaerythritol or triglycerol.

Preferably, the polyol is chosen from glycerol, diglycerol, pentaerythritol, sorbitol and mixtures thereof; and better still is pentaerythritol.

The polyol, or the polyol mixture, preferably represents 10 to 30% by weight, in particular 12 to 25% by weight, and even better 14 to 22% by weight, of the total weight of the final polycondensate.

Another constituent necessary for the preparation of the polycondensates according to the invention is a linear, branched and/or cyclic, saturated or unsaturated, non-aromatic monocarboxylic acid comprising 6 to 32 carbon atoms, in particular 8 to 28 carbon atoms and better still 10 to 24, or even 12 to 20, carbon atoms. It is quite obviously possible to use a mixture of such non-aromatic monocarboxylic acids.

The expression non-aromatic monocarboxylic acid is understood to mean a compound of formula RCOOH, in which R is a linear, branched and/or cyclic, saturated or unsaturated hydrocarbon radical comprising 5 to 31 carbon atoms, in particular 7 to 27 carbon atoms, and better still 9 to 23 carbon atoms, or even 11 to 19 carbon atoms.

Preferably, the R radical is saturated. Better still, the said R radical is linear or branched, and is preferably C5-C31, or even C11-C21.

In a particular embodiment of the invention, the non-aromatic monocarboxylic acid has a melting point greater than or equal to 25° C., in particular greater than or equal to 28° C., or even to 30° C.; it has in fact been observed that when such an acid is used, in particular in large quantity, it is possible, on the one hand, to obtain good gloss and staying power for the said gloss and, on the other hand, to reduce the quantity of waxes customarily present in the composition envisaged.

Among the non-aromatic monocarboxylic acids liable to be used, there may be mentioned, alone or as a mixture:
the saturated monocarboxylic acids such as caproic acid, caprylic acid, isoheptanoic acid, 4-ethylpentanoic acid, 2-ethylhexanoic acid, 4,5-dimethylhexanoic acid, 2-heptylheptanoic acid, 3,5,5-trimethylhexanoic acid, octanoic acid, isooctanoic acid, nonanoic acid, decanoic acid, isononanoic acid, lauric acid, tridecanoic acid, myristic acid, palmitic acid, stearic acid, isostearic acid, arachidic acid, behenic acid, cerotic(hexacosanoic) acid; cyclopentanecarboxylic acid, cyclopentaneacetic acid, 3-cyclopentylpropionic acid, cyclohexanecarboxylic acid, cyclohexylacetic acid, 4-cyclohexylbutyric acid;
unsaturated but non-aromatic monocarboxylic acids, such as caproleic acid, obtusilic acid, undecylenic acid, dodecylenic acid, linderic acid, myristoleic acid, physeteric acid, tsuzuic acid, palmitoleic acid, oleic acid, petroselinic acid, vaccenic acid, elaidic acid, gondoic acid, gadoleic acid, erucic acid, cetoleic acid, nervonic acid, linoleic acid, linolenic acid, arachidonic acid.

Among the non-aromatic monocarboxylic acids having a melting point greater than or equal to 25° C., there may be mentioned, alone or as a mixture:
among the saturated monocarboxylic acids: decanoic (capric) acid, lauric acid, tridecanoic acid, myristic acid, palmitic acid, stearic acid, arachidic acid, behenic acid, cerotic (hexacosanoic) acid;
among the unsaturated but non-aromatic monocarboxylic acids: petroselinic acid, vaccenic acid, elaidic acid, gondoic acid, gadoleic acid, erucic acid, nervonic acid.

Preferably, it is possible to use 2-ethylhexanoic acid, isooctanoic acid, lauric acid, myristic acid, isoheptanoic acid, isononanoic acid, nonanoic acid, palmitic acid, isostearic acid, stearic acid, behenic acid and mixtures thereof, and better still isostearic acid alone or stearic acid alone.

The said non-aromatic monocarboxylic acid, or the mixture of the said acids, preferably represents 30 to 80% by weight, in particular 40 to 75% by weight, or even 45 to 70% by weight, and even better still 50 to 65% by weight, of the total weight of the final polycondensate.

Another constituent necessary for the preparation of the polycondensates according to the invention is a polycarboxylic acid, and/or a cyclic anhydride of such a polycarboxylic acid, and/or a lactone carrying at least one COOH group; and mixtures thereof.

The said polycarboxylic acid may in particular be chosen from saturated or unsaturated, or even aromatic, linear, branched and/or cyclic polycarboxylic acids comprising 2 to 50, in particular 2 to 40, carbon atoms, in particular 3 to 36, or even 3 to 18, and better still 4 to 12 carbon atoms, or even 5 to 10 carbon atoms; the said acid comprising at least two carboxyl groups COOH, preferably 2 to 4 COOH groups; and capable of comprising 1 to 10, preferably 1 to 6, heteroatoms, which are identical or different, chosen from O, N and S; and/or capable of comprising at least one perfluorinated radical chosen from —CF₂— (divalent) or —CF₃.

Preferably, the said polycarboxylic acid is linear, saturated and aliphatic, and comprises 2 to 36 carbon atoms, in particular 3 to 18 carbon atoms, or even 4 to 12 carbon atoms; or else is aromatic and comprises 8 to carbon atoms. It preferably comprises 2 to 4 COOH groups.

The said cyclic anhydride of such a polycarboxylic acid may in particular correspond to one of the following formulae:

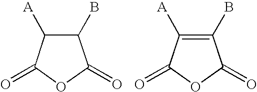

in which the A and B groups are, independently of each other:
a hydrogen atom,
a linear, branched and/or cyclic, saturated or unsaturated, aliphatic or aromatic carbon-containing radical comprising 1 to 16 carbon atoms, in particular 2 to 10 carbon atoms, or even 4 to 8 carbon atoms, in particular methyl or ethyl;
or else A and B taken together form a saturated or unsaturated, or even aromatic, ring comprising in total 5 to 14, in particular 5 to 10, or even 6 to 7 carbon atoms.

Preferably, A and B represent a hydrogen atom or form together an aromatic ring comprising in total 6 to 10 carbon atoms.

Among the polycarboxylic acids or their anhydrides, which are liable to be used, there may be mentioned, alone or as a mixture:
dicarboxylic acids such as decanedioic acid, dodecanedioic acid, cyclopropanedicarboxylic acid, cyclohexanedicarboxylic acid, cyclobutanedicarboxylic acid, 1,4-naphthalenedicarboxylic acid, 2,3-naphthalenedicarboxylic acid, 2,6-naphthalenedicarboxylic acid, suberic acid, oxalic acid, malonic acid, succinic acid, phthalic acid, terephthalic acid, isophthalic acid, tetrahydrophthalic acid, hexahydrophthalic acid, pimelic acid, sebacic acid, azelaic acid, glutaric acid, adipic acid, fumaric acid, maleic acid, itaconic acid, dimers of fatty acids (in particular as C36) such as the products marketed under the names Pripol 1006, 1009, 1013 and 1017, by Uniqema;
tricarboxylic acids such as cyclohexanetricarboxylic acid, trimellitic acid, 1,2,3-benzenetricarboxylic acid, 1,3,5-benzenetricarboxylic acid; aconitic, in particular trans-aconitic, acid;
tetracarboxylic acids such as butanetetracarboxylic acid and pyrromellitic acid,
the cyclic anhydrides of these acids and in particular phthalic anhydride, trimellitic anhydride, maleic anhydride and succinic anhydride.

Preferably, it is possible to use adipic acid, phthalic anhydride and/or isophthalic acid, and better still isophthalic acid alone.

There may also be mentioned the polycarboxylic acids chosen from, alone or as a mixture:
(i) polycarboxylic acids having a linear or branched, saturated or unsaturated chain, comprising at least one heteroatom chosen from O, N and/or S, in particular 1 to 10 heteroatoms which are identical or different, and/or comprising at least one perfluorinated radical —CF₂— or —CF₃ and having moreover at least 2 carboxyl groups COOH, in particular 2 to 4 COOH groups; and/or a cyclic anhydride of such a polycarboxylic acid;
   and/or
(ii) saturated or unsaturated, or even aromatic, heterocyclic polycarboxylic acids comprising at least one heteroatom chosen from O, N and/or S, in particular 1 to 10, or even 1 to 4, heteroatoms which are identical or different, and at least 2 carboxyl groups COOH, in particular 2 to 4 COOH groups; and/or a cyclic anhydride of such a polycarboxylic acid;
   and/or
(iii) polycarboxylic acids derived from sugar, which are liable to be obtained in particular by oxidation of an aldose, and comprising at least 2 carboxyl groups COOH, in particular 2 or 3 COOH groups; and/or a cyclic anhydride of such a polycarboxylic acid;
   and/or
(iv) itaconic anhydride and the 1,4-monoanhydride of 1,4,5,8-naphthalenetetracarboxylic acid;
   and/or
(v) polycarboxylic (including heterocyclic) amino acids, that is to say polycarboxylic acids having a linear, branched and/or cyclic, saturated or unsaturated chain optionally comprising at least one heteroatom chosen from O, N and/or S, in particular 1 to 10 heteroatoms which are identical or different, and/or optionally comprising at least one perfluorinated radical —CF₂— or —CF₃; and additionally comprising at least one primary, secondary or tertiary amine functional group (in particular NR1R2 with R1 and R2, independently of each other, chosen from H and C1-C12 alkyl), in particular 1 to 3 amine functional groups, which are identical or different, and having moreover at least 2 carboxyl groups COOH, in particular 2 to 4 COOH groups; and/or a cyclic anhydride of such a polycarboxylic acid.

There may be mentioned most particularly, alone or as a mixture, the following dicarboxylic acids:

(i)
2,2′-[1,5-pentanediylbis(thio)]bis-acetic acid
6,6′-[(1,2-dioxo-1,2-ethanediyl)diimino]bis-hexanoic acid
2,2′-sulphinylbis-acetic acid
4,13-dioxo-3,5,12,14-tetraazahexadecanedioic acid
poly(ethylene glycol)disuccinate, in particular having a mass of 250-600
poly(ethylene glycol)bis(carboxymethyl)ether, in particular having a mass of 250-600
poly[oxy(1,2-dicarboxy-1,2-ethanediyl)], in particular having a DP<10
8-[(carboxymethyl)amino]-8-oxooctanoic acid
2,2′-[methylenebis(sulphonyl)]bis-acetic acid
4,4′-(1,6-hexanediyldiimino)bis[4-oxo-butanoic] acid
4,9-dioxo-3,5,8,10-tetraazadodecanedioic acid
4-[(1-carboxyethyl)amino]-4-oxo-butanoic acid
6-[(3-carboxy-1-oxopropyl)amino]hexanoic acid
N,N′-(1,6-dioxo-1,6-hexanediyl)bis-glycine
N,N′-(1,6-dioxo-1,6-hexanediyl)bis-phenylalanine
N,N′-(1,3-dioxo-1,3-propanediyl)bis-glycine
4,4′-[(1,4-dioxo-1,4-butanediyl)diimino]bis-butanoic acid
4,4′-[(1,6-dioxo-1,6-hexanediyl)diimino]bis-butanoic acid
6,6′-[1,6-hexanediylbis(iminocarbonylimino)]bis-hexanoic acid
N-benzoyl-S-(carboxymethyl)-Cysteine
N,N′-(2,2,3,3-tetrafluoro-1,4-dioxo-1,4-butanediyl)bis-Glycine
N,N′-(2,2,3,3-tetrafluoro-1,4-dioxo-1,4-butanediyl)bis-Alanine
4,4′-[(2,2,3,3-tetrafluoro-1,4-dioxo-1,4-butanoic acid
N,N′-(1,5-dioxo-1,5-pentanediyl)bis-Glycine
N,N′-(1,9-dioxo-1,9-nonanediyl)bis-Glycine
N,N′-(1,10-dioxo-1,10-decanediyl)bis[N-methyl-Glycine
bis(3-carboxypropyl)ester of propanedioic acid
7,16-dioxo-6,8,15,17-tetraazadocosanedioic acid
N-benzoyl-N-(2-carboxyethyl)-Glycine
[2-[(2-carboxymethyl)amino]-2-oxoethyl]-benzenepropanoic acid
[2-[(2-carboxyethyl)amino]-2-oxoethyl]-benzenepropanoic acid

(ii)
4,7,9,12-tetraoxapentadecanedioic acid
2,3-pyridinedicarboxylic acid
4-pyranone-2,6-dicarboxylic acid
2,5-pyrazinedicarboxylic acid
2,5-pyridinedicarboxylic acid
2,3-benzofurandicarboxylic acid
7-oxabicyclo[2.2.1]heptane-2,3-dicarboxylic acid
3,4-pyridinedicarboxylic acid
2,4-pyridinedicarboxylic acid
3,5-pyridinedicarboxylic acid
2,6-pyridinedicarboxylic acid
1H-imidazole-4,5-dicarboxylic acid
2,3-quinolinedicarboxylic acid
6,6,7,7-tetrafluoro-3-oxabicyclo[3.2.0]heptane-2,4-dicarboxylic acid
2,6-pyrazinedicarboxylic acid
2,6-dimethyl-3,5-pyridinedicarboxylic acid
1-phenyl-1H-pyrazole-3,4-dicarboxylic acid
2,5-furandicarboxylic acid
3,4-furandicarboxylic acid
1,2,5-thiadiazole-3,4-dicarboxylic acid
1,4-dihydro-1,2,4,5-tetrazine-3,6-dicarboxylic acid
2,3-furandicarboxylic acid
3,4-thiophenedicarboxylic acid
1H-1,2,3-triazole-4,5-dicarboxylic acid
2-methylimidazole-4,5-dicarboxylic acid
2,4-quinolinedicarboxylic acid
naphtho[2,1-b]furan-1,2-dicarboxylic acid
3,4-quinolinedicarboxylic acid
7-oxabicyclo[2.2.1]hept-5-ene-2,3-dicarboxylic acid
2,3-quinoxalinedicarboxylic acid
1,4-piperazinedicarboxylic acid
2,5-dimethyl-3,4-furandicarboxylic acid
tetrahydro-2,5-thiophenedicarboxylic acid
4-phenyl-3,5-pyridinedicarboxylic acid
thieno[3,2-b]thiophene-2,5-dicarboxylic acid
3-methyl-2,4-thiophenedicarboxylic acid
naphthostyril-5,6-dicarboxylic acid
3-phenyl-2,4-quinolinedicarboxylic acid
3,4-dimethyl-2,5-dicarboxythiophene
3,4-diphenyl-2,5-thiophenedicarboxylic acid
2,5-diphenyl-3,4-furandicarboxylic acid
7-oxo-7H-benzimidazo[2,1-a]benz[de]isoquinoline-3,4-dicarboxylic acid
2,3-dihydro-1,3-dioxo-1H-benz[de]isoquinoline-6,7-dicarboxylic acid
3,4-bis(phenylmethoxy)-2,5-furandicarboxylic acid
4,4′-bibenzoic acid 2,2′-sulphone
2,7-diphenyl-m-anthrazoline-4,5-dicarboxylic acid
2,4-pyrimidinedicarboxylic acid
2-phenyl-4,5-thiazoledicarboxylic acid
6-phenyl-2,3-pyridinedicarboxylic acid
5,6-dimethyl-2,3-pyrazinedicarboxylic acid
3,7-dibenzothiophenedicarboxylic acid
9-oxo-9H-xanthene-1,7-dicarboxylic acid
2-(1,1-dimethylethyl)-H-imidazole-4,5-dicarboxylic acid
6,7-quinolinedicarboxylic acid
6-methyl-2,3-pyridinedicarboxylic acid
4,5-pyrimidinedicarboxylic acid
2-methyl-3,4-furandicarboxylic acid
1,2-indolizinedicarboxylic acid
2,8-dibenzothiophenedicarboxylic acid
3,6-pyridazinedicarboxylic acid
1,10-phenanthroline-2,9-dicarboxylic acid
1,4,5,6-tetrahydro-5,6-dioxo-2,3-pyrazine-dicarboxylic acid
3,4-dimethoxy-2,5-furandicarboxylic acid
2-ethyl-4,5-imidazoledicarboxylic acid
2-propyl-1H-imidazole-4,5-dicarboxylic acid
4-phenyl-2,5-pyridinedicarboxylic acid
4,5-pyridazinedicarboxylic acid
1,4,5,8-tetrahydro-1,4:5,8-diepoxynaphthalene-4a,8a-dicarboxylic acid
5,5-dioxide-2,8-dibenzothiophenedicarboxylic acid
pyrazolo[1,5-a]pyridine-2,3-dicarboxylic acid
2,3-dihydro-1H-pyrrolizine-1,7-dicarboxylic acid
6-methyl-2,4,5-pyridinetricarboxylic acid
pyrrolo[2,1,5-cd]indolizine-5,6-dicarboxylic acid
3,4-bis(2,2,3,3,4,4,4-heptafluorobutyl)-1H-pyrrole-2,5-dicarboxylic acid
6,7,9,10,17,18,20,21-octahydrodibenzo-[b,k][1,4,7,10,13,16]hexaoxacyclooctadecine-2,14-dicarboxylic acid
6,7,9,10,17,18,20,21-octahydrodibenzo-[b,k][1,4,7,10,13,16]hexaoxacyclooctadecine-2,13-dicarboxylic acid
2-methyl-3,4-quinolinedicarboxylic acid
4,7-quinolinedicarboxylic acid
3,5-isoxazoledicarboxylic acid
2-(trifluoromethyl)-3,4-furandicarboxylic acid
5-(trifluoromethyl)-2,4-furandicarboxylic acid
6-methyl-2,4-quinolinedicarboxylic acid
5-oxo-1,2-pyrrolidinedicarboxylic acid
5-ethyl-2,3-pyridinedicarboxylic acid
1,2-dihydro-2-oxo-3,4-quinolinedicarboxylic acid
4,6-phenoxathiindicarboxylic acid
10,10-dioxide 1,9-phenoxathiindicarboxylic acid
3,4-dihydro-2H-1,4-thiazine-3,5-dicarboxylic acid
2,7-di(tert-butyl)-9,9-dimethyl-4,5-xanthene-dicarboxylic acid
6-methyl-2,3-quinoxalinedicarboxylic acid
3,7-quinolinedicarboxylic acid
2,5-quinolinedicarboxylic acid
2-methyl-6-phenyl-3,4-pyridinedicarboxylic acid
3,4-dimethylthieno[2,3-b]thiophene-2,5-dicarboxylic acid
3,4-dimethoxythiophene-2,5-dicarboxylic acid
5-methyl-3,4-isoxazoledicarboxylic acid
2,6-bis(aminocarbonyl)-3,5-pyridinedicarboxylic acid
3,5-bis(aminocarbonyl)-2,6-pyrazinedicarboxylic acid
2,3-pyridinedicarboxylic acid
6-(1,1-dimethylethyl)-2-ethyl-3,4-pyridine-dicarboxylic acid
3-methyl-5-phenyl-2,4-thiophenedicarboxylic acid
1,2-dihydro-2-oxo-6-phenyl-3,5-pyridinedicarboxylic acid
8-methyl-2,4-quinolinedicarboxylic acid
4-ethyl-2,6-dimethyl-3,5-pyridinedicarboxylic acid
5-(phenoxymethyl)-2,4-furandicarboxylic acid
5-(acetylamino)-3-methyl-2,4-thiophenedicarboxylic acid
2-(4-heptylphenyl)-4,8-quinolinedicarboxylic acid
2,8-bis(4-heptylphenyl)pyrido[3,2-g]quinoline-4,6-dicarboxylic acid
1,2,3,4,6,7,8,9-octahydro-2,8-dioxopyrido[3,2]-quinoline-3,7-dicarboxylic acid
2,8-dimethylpyrido[3,2-g]quinoline-3,7-dicarboxylic acid
5,6-quinolinedicarboxylic acid
6-ethyl-2-methylcinchomeronic acid
2-methyl-6-propylcinchomeronic acid
6-isopropyl-2-methylcinchomeronic acid
6-tert-butyl-2-methylcinchomeronic acid
1,4-dimethyl-7-oxabicyclo[2.2.1]heptane-2,3-dicarboxylic acid
1,2-dihydro-2-oxo-3,8-quinolinedicarboxylic acid
1,2-dihydro-2-oxo-3,6-quinolinedicarboxylic acid
1,2-dihydro-2-oxo-3,7-quinolinedicarboxylic acid
3,7-dimethyl-2,8-diphenylpyrido[3,2-g]quinoline-4,6-dicarboxylic acid
8-methyl-2,3-quinolinedicarboxylic acid
3-[[(1,1-dimethylethyl)amino]sulphonyl]-2,5-thiophenedicarboxylic acid
4-(acetylamino)-2,3-thiophenedicarboxylic acid
2,5-pyridinedicarboxylic acid
2,6-pyridinedicarboxylic acid
2,4-thiophenedicarboxylic acid
2,5-thiophenedicarboxylic acid
1,4-pyran-2,6-dicarboxylic acid

(iii)
ribaric acid
glucaric acid
xylaric acid
arabinaric acid
mannaric acid
idaric acid
altraric acid
L-glucaric acid
L-arabinaric acid
allaric acid
galactaric acid
meso-tartaric acid
D-glucaric acid
L-idaric acid
hexaric acid
2,3-dihydroxybutanedioic acid
D-tartaric acid
DL-tartaric acid
D-glucaric acid
tartaric acid
tetrahydroxysuccinic acid
2-carboxy-2,3-dideoxy-D-manno-2-octulopyranosonic acid
methyl 3-deoxy-D-arabino-2-heptulopyranosaric acid
D-lyxo-2-heptulopyranosaric acid
2,6-anhydro-L-glycero-L-galactoheptaric acid

(iv)
1,4,5,8-naphthalenetetracarboxylic acid 1,4-mono-anhydride
itaconic anhydride

(v)
1,4-dihydro-4-oxo-2,6-pyridinedicarboxylic acid
2,6-piperidinedicarboxylic acid
1H-pyrrole-3,4-dicarboxylic acid
4-amino-2,6-dicarboxylic acid
1-methyl-1H-pyrazole-3,4-dicarboxylic acid
2,3-piperidinedicarboxylic acid
1-methyl-1H-imidazole-4,5-dicarboxylic acid
2,4-thiazolidinedicarboxylic acid
1-(phenylmethyl)-1H-imidazole-4,5-dicarboxylic acid
5-amino-6-oxo-2,3-piperidinedicarboxylic acid
5-amino-6-oxo-2,4-piperidinedicarboxylic acid
5-amino-6-oxo-[2S-(2α,4β,5α)]-2,4-piperidine-dicarboxylic acid
(2S,4R)-2,4-pyrrolidinedicarboxylic acid
(2S-cis)-2,4-pyrrolidinedicarboxylic acid
2-amino-1H-imidazole-4,5-dicarboxylic acid
2,5-pyrrolidinedicarboxylic acid
4-amino-3,5-isothiazoledicarboxylic acid
1-methyl-1H-pyrazole-3,5-dicarboxylic acid
7-(diethylamino)-2-oxo-2H-1-benzopyran-3,4-dicarboxylic acid
3,4-diethyl-1H-pyrrole-2,5-dicarboxylic acid
1-phenyl-1H-pyrrole-3,4-dicarboxylic acid
cis-2,3-piperazinedicarboxylic acid
2,3-piperazinedicarboxylic acid
2,5-piperazinedicarboxylic acid
2,6-piperazinedicarboxylic acid
2-amino-3,5-pyridinedicarboxylic acid
2-methylpyrrole-3,4-dicarboxylic acid
4-(methylamino)-2,6-pyridinedicarboxylic acid
2-amino-6-methyl-3,4-pyridinedicarboxylic acid
5-amino-2-methyl-3,4-pyridinedicarboxylic acid
2-amino-6-methyl-3,5-pyridinedicarboxylic acid
2,5-dimethylpyrrole-3,4-dicarboxylic acid
2-amino-6-hydroxy-3,5-pyridinedicarboxylic acid
2,4-pyrrolidinedicarboxylic acid
1H-indole-2,4-dicarboxylic acid
1H-indole-2,6-dicarboxylic acid
1H-indole-2,5-dicarboxylic acid
5-phenyl-2,4-pyrrolidinedicarboxylic acid
5-methyl-2,4-pyrrolidinedicarboxylic acid
trans-2,4-azetidinedicarboxylic acid
cis-2,4-azetidinedicarboxylic acid
3,5-piperidinedicarboxylic acid
2,3-pyrrolidinedicarboxylic acid
2,3-azetidinedicarboxylic acid
3,4-pyrrolidinedicarboxylic acid
2,3-dihydro-6H-1,4-dioxino[2,3-c]pyrrole-5,7-dicarboxylic acid
1H-imidazole-2,4-dicarboxylic acid
1-butyl-1H-pyrrole-2,3-dicarboxylic acid
3-amino-1-oxide-2,4-pyridinedicarboxylic acid
2,3-dihydro-5-phenyl-1H-pyrrolizine-6,7-dicarboxylic acid
3a,4,5,9b-tetrahydro-3H-cyclopenta[c]quinoline-4,6-dicarboxylic acid
3a,4,5,9b-tetrahydro-3H-cyclopenta[c]quinoline-4,8-dicarboxylic acid
2,3-dihydro-1H-imidazole-4,5-dicarboxylic acid
5-amino-6-methyllutidinic acid
1H-indole-3,7-dicarboxylic acid
3,3-dimethyl-2,6-piperidinedicarboxylic acid
1-butyl-2,5-pyrrolidinedicarboxylic acid
1H-indole-4,6-dicarboxylic acid
1-(phenylmethyl)-3,4-pyrrolidinedicarboxylic acid
3-(carboxymethyl)-1H-indole-2,6-dicarboxylic acid
3,4-bis(2,2,2-trifluoroethyl)-1H-pyrrole-2,5-dicarboxylic acid
9-hexyl-9H-carbazole-3,6-dicarboxylic acid
3-methyl-5-(1-piperazinylsulphonyl)-2,4-thiophene-dicarboxylic acid
2,3,4,9-tetrahydro-1H-carbazole-5,7-dicarboxylic acid
2,3-dimethyl-1H-indole-4,6-dicarboxylic acid
7-amino-1,4-dihydro-4-oxo-3,6-quinolinedicarboxylic acid
5-amino-3-methyl-2,4-thiophenedicarboxylic acid
(m-tolylimino)diacetic acid
(o-tolylimino)diacetic acid
D-cystathionine
phenethyliminodiacetic acid
2-benzyl-2,2′-iminodiacetic acid
L-α-glutamyl-L-alanyl-L-alanine
N,N′-dibenzylethylenediaminediacetic acid
N-L-γ-glutamyl-D-alanine
glycyl-L-glutamylglycine
N-(carboxymethyl)-N-(tetrahydro-1,1-dioxido-3-thienyl)glycine
N-(2-carboxyethyl)-N-phenyl-β-alanine
N-(carboxymethyl)-N-octylglycine
N-(tert-butoxycarbonyl)iminodiacetic acid
N-(carboxymethyl)-L-alanine
N-(6-aminohexyl)-N-(carboxymethyl)glycine
N-(carboxymethyl)-N-tetradecylglycine
N-(1-carboxyethyl)-D-alanine
N-(carboxymethyl)-D-alanine
decyliminodiacetic acid
3,3′-(dimethylhydrazono)bispropanoic acid
N-(carboxymethyl)-N-[2-(2,6-dioxo-4-morpholinyl)-ethyl]glycine
N-α-aspartylglycine
N-β-aspartylglycine
N-L-α-aspartyl-β-alanine
3,4-xylylamino-N,N-diacetic acid
N-(1-carboxyethyl)alanine
N-(carboxymethyl)alanine
N,N′-methylenebisglycine
N-(aminomethyl)-N-(carboxymethyl)glycine
2,2′-(methylhydrazono)bisacetic acid
N-(2-carboxyethyl)-N-(4-methylphenyl)-β-alanine
N-(2-carboxyethyl)-N-(3-methylphenyl)-β-alanine
3-[(carboxymethyl)amino]alanine
D-α-aspartyl-D-alanine
N-(2-carboxyethyl)-N-(1-oxohexadecyl)-β-alanine
N-(2-carboxyethyl)-N-(1-oxodecyl)-β-alanine
N-(2-carboxyethyl)-N-(1-oxotetradecyl)-β-alanine
amino[(carboxymethyl)thio]acetic acid
N,N′-1,6-hexanediylbis-β-alanine
N-(carboxymethyl)-N-phenyl-β-alanine
N-(1-carboxyethyl)-L-alanine
L-glutamic acid
L-aspartic acid

There may also be mentioned, alone or as a mixture, the following tricarboxylic and tetracarboxylic acids, and their anhydrides:
3,3′,3″-[1,2,3-propanetriyltris(oxy)tris-propanoic acid
pyrazinetricarboxylic acid
4-(3-carboxyphenyl)-2,5-pyridinedicarboxylic acid
3-(carboxymethyl)-2,4-quinolinedicarboxylic acid
3-(carboxymethyl)-1H-indole-2,5-dicarboxylic acid
3-C-carboxy-2-deoxy-D-threopentaric acid
hydroxycitric acid
D-glucopyranuronosyl-D-arabino-2-hexulofuranosidaric acid
2,3,5,6-pyridinetetracarboxylic acid
N,N′-1,2-ethanediylbis[N-(carboxymethyl)-β-alanine
L-α-aspartyl-L-aspartic acid
4-[bis(carboxymethyl)amino]benzoic acid
7-[bis(carboxymethyl)amino]heptanoic acid
N-(2-carboxyethyl)aspartic acid
3-[bis(2-carboxyethyl)amino]benzoic acid
4-[bis(2-carboxyethyl)amino]benzoic acid.

Preferably, it is possible to use 6,6′-[(1,2-dioxo-1,2-ethanediyl)diimino]bis-hexanoic acid, 2,2′-sulphinylbis-acetic acid, 4,13-dioxo-3,5,12,14-tetra-azahexadecanedioic acid, poly(ethylene glycol)-disuccinate, poly(ethylene glycol)bis(carboxymethyl)-ether, 8-[(carboxymethyl)amino]-8-oxo-octanoic acid, 2,2′-[methylenebis(sulphonyl)]bis-acetic acid, 4,4′-(1,6-hexanediyldiimino)bis[4-oxo-butanoic] acid, 4,9-dioxo-3,5,8,10-tetraazadodecanedioic acid, 4-[(1-carboxyethyl)amino]-4-oxo-butanoic acid, 6-[(3-carboxy-1-oxopropyl)amino]hexanoic acid, N,N′-(1,6-dioxo-1,6-hexanediyl)bis-glycine, N,N′-(1,3-dioxo-1,3-propane-diyl)bis-glycine, 4,7,9,12-tetraoxapentadecanedioic acid, 4-pyranone-2,6-dicarboxylic acid, 2,5-pyrazinedicarboxylic acid, 1H-imidazole-4,5-dicarboxylic acid, 2,6-pyrazinedicarboxylic acid, 2,5-furandicarboxylic acid, 3,4-furandicarboxylic acid, 2,3-furandicarboxylic acid, 2,5-diphenyl-3,4-furandicarboxylic acid, 2-methyl-3,4-furandicarboxylic acid, D-tartaric acid, DL-tartaric acid, L-tartaric acid, galactaric acid, D-glucaric acid, 2,5-pyridinedicarboxylic acid, 2,5-pyrrolidinedicarboxylic acid, 1-phenyl-1H-pyrrole-3,4-dicarboxylic acid, 2,4-pyrrolidinedicarboxylic acid, 5-phenyl-2,4-pyrrolidinedicarboxylic acid, 3,5-piperidinedicarboxylic acid, 3,4-pyrrolidinedicarboxylic acid, 1-butyl-2,5-pyrrolidinedicarboxylic acid, 1-(phenylmethyl)-3,4-pyrrolidinedicarboxylic acid, N-(2-carboxyethyl)-N-phenyl-β-Alanine, N-(carboxymethyl)-N-octyl-glycine, N-(1-carboxyethyl)-L-Alanine, L-glutamic acid, L-aspartic acid, N-(2-carboxyethyl)-Aspartic acid; and mixtures thereof.

Most particularly preferred are 2,2′-sulphinylbis-acetic acid, 2,2′-[methylenebis(sulphonyl)]bis-acetic acid, N,N′-(1,3-dioxo-1,3-propanediyl)bis-glycine, 2,5-furandicarboxylic acid, D-tartaric acid, DL-tartaric acid, L-tartaric acid, galactaric acid, L-glutamic acid, L-aspartic acid, and mixtures thereof.

It is also possible to use a lactone comprising at least one carboxyl group, in particular 1, 2 or 3 COOH groups. Preferably, the lactones comprise 5 to 14 carbon atoms, in particular 6 to 13, or even 6 to 12 carbon atoms.

The following lactones may be most particularly mentioned, alone or as a mixture:
tetrahydro-2,2-dimethyl-5-oxo-3-furancarboxylic acid
4,7,7-trimethyl-3-oxo-2-oxabicyclo[2.2.1]heptane-1-carboxylic acid
4,6-dimethyl-2-oxo-2H-pyran-5-carboxylic acid
2-oxo-2H-pyran-5-carboxy-2-pentenedioic acid
2-oxo-2H-1-benzopyran-3-carboxylic acid
2-oxo-2H-pyran-6-carboxylic acid
1,3-dihydro-3-oxo-1-isobenzofurancarboxylic acid
4-methyl-2-oxo-2H-1-benzopyran-3-carboxylic acid
1-oxo-1H-2-benzopyran-3-carboxylic acid
8-methoxy-2-oxo-2H-1-benzopyran-3-carboxylic acid
2-oxo-1-oxaspiro[4.5]decane-4-carboxylic acid
2-oxo-2H-pyran-3-carboxylic acid
4-methyl-2-oxo-2H-pyran-6-carboxylic acid
3-oxo-3H-naphtho[2,1-b]pyran-2-carboxylic acid
tetrahydro-5-oxo-2,3-furandicarboxylic acid
1,3-dihydro-3-oxo-4-isobenzofurancarboxylic acid
1,3-dihydro-1-oxo-5-isobenzofurancarboxylic acid
hexahydro-2-oxo-3,5-methano-2H-cyclopenta[b]furan-7-carboxylic acid
6-methyl-2,4-dioxo-2H-pyran-5-carboxylic acid
1-oxo-3-isochromancarboxylic acid
2-oxo-2H-1-benzopyran-6-carboxylic acid
6-methyl-2-oxo-2H-1-benzopyran-3-carboxylic acid
2,5-dihydro-4,5,5-trimethyl-2-oxo-3-furancarboxylic acid
tetrahydro-5-oxo-2-phenyl-3-furancarboxylic acid
tetrahydro-5-oxo-4-propyl-2-furoic acid
2-butyl-2,3-dideoxypentaric acid
2-oxo-2H-1-benzopyran-7-carboxylic acid
2-oxo-1-oxaspiro[4.4]nonane-4-carboxylic acid
4-ethyltetrahydro-5-oxo-2-furoic acid
5-ethyltetrahydro-2,3-dimethyl-6-oxo-2H-pyran-2-carboxylic acid
7-methoxy-2-oxo-2H-1-benzopyran-3-carboxylic acid
2-oxo-2H-1-benzopyran-4-carboxylic acid
2-oxo-6-pentyl-2H-pyran-3-carboxylic acid
7-oxo-4-oxepanecarboxylic acid
3-(carboxymethyl)-2,3-dideoxypentaric acid
2,3-dihydro-2-oxo-7-benzofurancarboxylic acid
1,3,4,5-tetrahydro-1-oxo-2-benzoxepin-7-carboxylic acid
3,4-dihydro-3-oxo-1H-2-benzopyran-6-carboxylic acid
2,3,4,5-tetrahydro-2-oxo-1-benzoxepin-7-carboxylic acid
3,4-dihydro-1-oxo-1H-2-benzopyran-8-carboxylic acid
1,3,4,5-tetrahydro-3-oxo-2-benzoxepin-9-carboxylic acid
1,3,4,5-tetrahydro-3-oxo-2-benzoxepin-7-carboxylic acid
3,4-dihydro-2-oxo-2H-1-benzopyran-8-carboxylic acid
1,3,4,5-tetrahydro-1-oxo-2-benzoxepin-9-carboxylic acid
3,4-dihydro-1-oxo-1H-2-benzopyran-6-carboxylic acid
3,4-dihydro-3-oxo-1H-2-benzopyran-8-carboxylic acid
2,3,4,5-tetrahydro-2-oxo-1-benzoxepin-9-carboxylic acid
isocitric acid lactone
5-oxo-2-tetrahydrofurancarboxylic acid.

Preferably, it is possible to use tetrahydro-5-oxo-2,3-furandicarboxylic acid, 1,3-dihydro-3-oxo-4-isobenzofurancarboxylic acid, 1,3-dihydro-1-oxo-5-isobenzofurancarboxylic acid, tetrahydro-5-oxo-2-phenyl-3-furancarboxylic acid, isocitric acid lactone, 5-oxo-2-tetrahydrofurancarboxylic acid and mixtures thereof.

The said polycarboxylic acid and/or its cyclic anhydride and/or the lactone, and mixtures thereof, preferably represents 1 to 40% by weight, in particular to 30% by weight, and even better 10 to 25% by weight, of the total weight of the final polycondensate.

The polycondensate according to the invention may additionally comprise a silicone having a hydroxyl (OH) and/or carboxyl (COOH) functional group. It may comprise 1 to 3 hydroxyl and/or carboxyl functional groups, and preferably comprises two hydroxyl functional groups or else two carboxyl functional groups. These functional groups may be located at the chain end or in the chain, but advantageously at the chain end.

Use is preferably made of silicones having a weight-average molecular mass (Mw) between 300 and 20 000, in particular 400 and 10 000, or even 800 and 4000.

This silicone may be of formula:

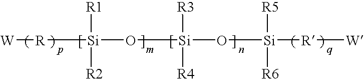

in which:
W and W′ are, independently of each other, OH or COOH; preferably W═W′;
p and q are, independently of each other, equal to 0 or 1,
R and R′ are, independently of each other, a saturated or unsaturated, or even aromatic, linear, branched and/or cyclic divalent carbon, in particular hydrocarbon, radical comprising 1 to 12 carbon atoms, in particular 2 to 8 carbon atoms, and optionally additionally comprising one or more heteroatoms chosen from O, S and N, in particular O (ether);
   in particular R and/or R′ may be of formula —(CH₂)ₐ— with a=1-12, in particular methylene, ethylene, propylene, phenylene;

or else of formula —[(CH₂)ₓO]_{z}— with x=1, 2 or 3 and z=1-10; in particular x=2 or 3 and z=1-4; and even better x=3 and z=1,
R1 to R6 are, independently of each other, a saturated or unsaturated or even aromatic linear, branched and/or cyclic carbon radical comprising 1 to 20 carbon atoms, in particular 2 to 12 carbon atoms; preferably R1 to R6 are saturated or else aromatic, and may in particular be chosen from alkyl radicals, in particular methyl, ethyl, propyl, isopropyl, butyl, pentyl, hexyl, octyl, decyl, dodecyl and octadecyl radicals, cycloalkyl radicals, in particular cyclohexyl radical, aryl radicals, in particular phenyl and napthyl, arylalkyl radicals, in particular benzyl and phenylethyl and tolyl and xylyl radicals,
m and n are, independently of each other, integers between 1 and 140, and are such that the weight-average molecular mass (Mw) of the silicone is between 300 and 20 000, in particular between 400 and 10 000, or even between 800 and 4 000.

Mention may be made in particular of α,ω-diol or α,ω-dicarboxylic polyalkylsiloxanes, and in particular α,ω-diol polydimethylsiloxanes and α,ω-dicarboxylic polydimethylsiloxanes; α,ω-diol or α,ω-dicarboxylic polyarylsiloxanes and in particular α,ω-diol or α,ω-di-carboxylic polyphenylsiloxanes; polyarylsiloxanes containing silanol functional groups such as polyphenylsiloxane; polyalkylsiloxanes containing silanol functional groups such as polydimethylsiloxane; polyaryl/alkylsiloxanes containing silanol functional groups such as polyphenyl/methylsiloxane or else polyphenyl/propylsiloxane.

Most particularly, use will be made of α,ω-diol polydimethylsiloxanes having a weight-average molecular mass (Mw) between 400 and 10 000, or even between 500 and 5000, and in particular between 800 and 4000.

When it is present, the said silicone may preferably represent 0.1 to 15% by weight, in particular 1 to 10% by weight, or even 2 to 8% by weight, of the weight of the polycondensate.

The polycondensate according to the invention does not comprise other families of monomers than those mentioned above; in particular, it does not comprise aromatic monocarboxylic acids. The fact that the polycondensate is liable to be obtained by reaction of the sole monomers cited above does not exclude, quite obviously, the presence, during the reaction, of a catalyst, a primer or any other customary adjuvant in esterification and polymerization reactions.

Preferably, the polycondensate according to the invention has:
an acid value, expressed in mg of potassium hydroxide per g of polycondensate, greater than or equal to 1; in particular between 2 and 30, and better still between 2.5 and 15; and/or
a hydroxyl value, expressed in mg of potassium hydroxide per g of polycondensate, greater than or equal to 40; in particular between 40 and 120, and better still between 45 and 80.

These acid and hydroxyl values can be easily determined by persons skilled in the art by the usual analytical methods.

Preferably, the polycondensate according to the invention has a viscosity, measured at 110° C., between and 4000 mPa·s, in particular between 30 and 3500 mPa·s, or even between 40 and 3000 mPa·s, and better still between 50 and 2500 mPa·s. This viscosity is measured in the manner described before the example.

Preferably, the polycondensate according to the invention has a weight-average molecular mass (Mw) between 1500 and 300 000, or even between 2000 and 200 000, and in particular between 3000 and 150 000.

The average molecular weight may be determined by gel permeation chromatography or by light scattering, depending on the solubility of the polymer considered.

One of the important characteristics of the polymer according to the invention is that it has a very high viscosity, which may be characterized as follows.

At 25° C., when this viscosity can be measured (in particular when the polymer is not in solid form), it is strictly greater than 5000 mPa·s (centipoises), in particular between 5100 and 10 000 mPa·s, or even between 6000 and 9500 mPa·s.

Preferably, the polycondensate according to the invention has a viscosity, measured at 110° C., between 100 and 4000 mPa·s, in particular between 130 and 3500 mPa·s, or even between 150 and 3000 mPa·s and better still between 200 and 2500 mPa·s.

Preferably still, the polycondensate according to the invention has a viscosity, measured at 80° C., between 150 and 5000 mPa·s, in particular between 200 and 4000 mPa·s, or even between 250 and 3000 mPa·s and better still between 300 and 2000 mPa·s

This viscosity may be measured with the aid of a cone-plate viscometer of the Brookfield CAP 1000+ type. The appropriate cone-plate is determined by persons skilled in the art on the basis of their knowledge; in particular:
between 50 and 500 mPa·s, it is possible to use a cone 02
between 500 and 1000 mPa·s: cone 03
between 1000 and 4000 mPa·s: cone 05
between 4000 and 10 000 mPa·s: cone 06.

Moreover, the polycondensate is advantageously soluble in the cosmetic oily media customarily used, and in particular in vegetable oils, alkanes, fatty esters, fatty alcohols, silicone oils, and more particularly in media comprising isododecane, Parleam, isononyl isononanoate, octyldodecanol, phenyl trimethicone, C12-C15 alkyl benzoate and/or D5 (decamethyl-cyclopentasiloxane.

The expression soluble is understood to mean that the polymer forms a clear solution in at least one solvent chosen from isododecane, Parleam, isononyl isononanoate, octyldodecanol and C12-C15 alkyl benzoate, in an amount of at least 50% by weight, at 70° C. Some compounds even have a particularly advantageous solubility in certain fields of application, namely a solubility in at least one of the solvents mentioned above, in an amount of at least 50% by weight at 25° C.

The polycondensate according to the invention may be prepared by the esterification/polycondensation methods customarily used by persons skilled in the art. By way of illustration, a general method of preparation consists:
in mixing the polyols and the non-aromatic monocarboxylic acids,
in heating the mixture under an inert atmosphere, first up to the melting point (generally 100-130° C.) and then at a temperature between 150 and 220° C. until there is complete consumption of the monocarboxylic acids (obtained when the acid value is less than or equal to 1), preferably while continuously distilling the water formed, and then
in optionally cooling the mixture to a temperature of between 90 and 150° C.,
in adding the polycarboxylic acids and/or the cyclic anhydrides and/or the lactones, and optionally the silicones having hydroxyl or carboxyl functional groups, all at once or sequentially, and then
in again heating to a temperature less than or equal to 220° C., in particular between 170 and 220° C., preferably while continuing to remove the water formed, until the desired characteristics in terms of acid value, viscosity, hydroxyl value and solubility are obtained.

It is possible to add conventional esterification catalysts, for example of the sulphonic acid type (in particular at a concentration by weight between 1 and 10%) or of the titanate type (in particular at a concentration by weight between 5 and 100 ppm). It is also possible to carry out the reaction, entirely or partially, in an inert solvent such as xylene and/or under reduced pressure, in order to facilitate the removal of water. Advantageously, neither catalyst nor solvent is used.

The said method of preparation may additionally comprise a step of adding at least one antioxidant to the reaction medium, in particular at a concentration by weight between 0.01 and 1%, relative to the total weight of monomers, so as to limit the possible degradations linked to prolonged heating. The antioxidant may be of the primary type or of the secondary type, and may be chosen from hindered phenols, aromatic secondary amines, organophosphate compounds, sulphur compounds, lactones, acrylated bisphenols; and mixtures thereof. Among the antioxidants particularly preferred, there may be mentioned in particular BHT, BHA, TBHQ, 1,3,5-trimethyl-2,4,6-tris(3,5-di-tert-butyl-4-hydroxy-benzyl)benzene, octadecyl 3,5-di-tert-butyl-4-hydroxy-cinnamate, tetrakis-methylene-3-(3,5-di-tert-butyl-4-hydroxyphenyl)propionate methane, octadecyl 3-(3,5-di-tert-butyl-4-hydroxyphenyl)propionate, 2,5-di-tert-butylhydroquinone, 2,2-methybis(4-methyl-6-tert-butylphenol), 2,2-methylenebis(4-ethyl-6-tert-butylphenol), 4,4-butylidenebis(6-tert-butyl-m-cresol), N,N-hexamethylene bis(3,5-di-tert-butyl-4-hydroxyhydrocinnamamide), pentaerythritol tetrakis(3-(3,5-di-tert-butyl-4-hydroxyphenyl)propionate), in particular that marketed by CIBA under the name IRGANOX 1010; octadecyl 3-(3,5-di-tert-butyl-4-hydroxyphenyl)propionate, in particular that marketed by CIBA under the name IRGANOX 1076; 1,3,5-tris(3,5-di-tert-butyl-4-hydroxybenzyl)-1,3,5-triazine-2,4,6-(1H,3H,5H)trione, in particular that marketed by Mayzo of Norcross, Ga. under the name BNX 3114; distearyl pentaerythritol diphosphite, tris(2,4-di-tert-butylphenyl)phosphite, in particular that marketed by CIBA under the name IRGAFOS 168; dilauryl thiodipropionate, in particular that marketed by CIBA under the name IRGANOX PS800; bis(2,4-di-tert-butyl)pentaerythritol diphosphite, in particular that marketed by CIBA under the IRGAFOS 126; bis(2,4-bis[2-phenylpropan-2-yl]phenyl)pentaerythritol diphosphite, triphenylphosphite, (2,4-di-tert-butylphenyl)pentaerythritol diphosphite, in particular that marketed by GE Speciality Chemicals under the name ULTRANOX 626; tris(nonylphenyl)phosphite, in particular that marketed by CIBA under the name IRGAFOS TNPP; 1:1 mixture of N,N-hexamethylenebis(3,5-di-tert-butyl-4-hydroxyhydrocinnamamide) and of tris(2,4-di-tert-butylphenyl)phosphate, in particular that marketed by CIBA under the name Irganox B 1171; tetrakis(2,4-di-tert-butylphenyl)phosphite, in particular that marketed by CIBA under the name IRGAFOS P-EPQ; distearyl thiodipropionate, in particular that marketed by CIBA under the name IRGANOX PS802; 2,4-bis(octylthiomethyl)-o-cresol, in particular that marketed by CIBA under the name IRGANOX 1520; 4,6-bis(dodecylthiomethyl)-o-cresol, in particular that marketed by CIBA under the name IRGANOX 1726.

The polycondensates according to the invention may be used very advantageously in a composition, in particular a cosmetic or pharmaceutical composition, which comprises, moreover, a physiologically, in particular cosmetically or pharmaceutically, acceptable medium, that is to say a medium that is compatible with the cutaneous tissues such as the skin of the face or of the body, and keratin materials such as the hair, the eyelashes, the eyebrows of the nails.

The quantity of polycondensate present in the compositions of course depends on the type of composition and on the desired properties and may vary within a very broad range, generally between 0.1 and 70% by weight, preferably between 1 and 50% by weight, in particular between 10 and 45% by weight, or even between 20 and 40% by weight, and even better between and 35% by weight, relative to the weight of the final cosmetic or pharmaceutical composition.

The composition may then comprise, depending on the application envisaged, the usual constituents for this type of composition.

The composition according to the invention may advantageously comprise a liquid fatty phase, which may constitute a solvent medium for the polymers according to the invention, and which may comprise at least one compound chosen from volatile or non-volatile, carbonaceous, hydrocarbon, fluorinated and/or silicone oils and/or solvents of inorganic, animal, plant or synthetic origin, alone or as a mixture insofar as they form a homogeneous and stable mixture and are compatible with the use envisaged.

The expression “volatile” is understood to mean, for the purposes of the invention, any compound liable to evaporate on contact with keratin materials, or the lips, in less than one hour, at room temperature (25° C.) and atmospheric pressure (1 atm). In particular, this volatile compound has a non-zero vapour pressure, at room temperature and atmospheric pressure, in particular ranging from 0.13 Pa to 40 000 Pa (10³ to 300 mmHg), in particular ranging from 1.3 Pa to 13 000 Pa (0.01 to 100 mmHg), and more particularly ranging from 1.3 Pa to 1300 Pa (0.01 to 10 mmHg). By contrast, the expression “non-volatile” is understood to mean a compound which remains on the keratin materials or the lips at room temperature and atmospheric pressure for at least one hour and which has in particular a vapour pressure of less than 10⁻³ mmHg (0.13 Pa).

Preferably, the physiologically acceptable medium of the composition according to the invention may comprise, in a liquid fatty phase, at least one oil and/or solvent which may be chosen from, alone or as a mixture:

1/Esters of monocarboxylic acids with monoalcohols and polyalcohols; advantageously, the said ester is a C12-C15 alkyl benzoate or corresponds to the following formula: R′₁—COO—R′₂ where:

R′₁ represents a linear or branched alkyl radical of 1 to 40 carbon atoms, preferably of 7 to 19 carbon atoms, optionally comprising one or more ethylenic double bonds, optionally substituted and whose hydrocarbon chain may be interrupted by one or more heteroatoms chosen from N and O and/or one or more carbonyl functional groups, and

R′₂ represents a linear or branched alkyl radical of 1 to 40 carbon atoms, preferably of 3 to 30 carbon atoms and even better of 3 to 20 carbon atoms, optionally comprising one or more ethylenic double bonds, optionally substituted and whose hydrocarbon chain may be interrupted by one or more heteroatoms chosen from N and O and/or one or more carbonyl functional groups.

The expression “optionally substituted” is understood to mean that R′₁ and/or R′₂ may carry one or more substituents chosen, for example, from the groups comprising one or more heteroatoms chosen from O and/or N, such as amino, amine, alkoxy, hydroxyl.

Examples of R′₁ groups are those derived from fatty acids, preferably higher fatty acids, chosen from the group constituted of acetic, propionic, butyric, caproic, caprylic, pelargonic, capric, undecanoic, lauric, myristic, palmitic, stearic, isostearic, arachidic, behenic, oleic, linolenic, linoleic, oleostearic, arachidonic and erucic acids, and mixtures thereof. Preferably, R′₁ is an unsubstituted branched alkyl group of 4 to 14 carbon atoms, preferably of 8 to carbon atoms and R₂ is an unsubstituted branched alkyl group of 5 to 15 carbon atoms, preferably of 9 to 11 carbon atoms.

There may preferably be mentioned in particular C₈-C₄₈ esters, optionally incorporating in their hydrocarbon chain one or more heteroatoms among N and O and/or one or more carbonyl functional groups; and more particularly purcellin oil (cetostearyl octanoate), isononyl isononanoate, isopropyl myristate, isopropyl palmitate, 2-ethylhexyl palmitate, 2-octyldodecyl stearate, 2-octyldodecyl erucate, isostearyl isostearate, C₁₂ to C₁₅ alcohol benzoate, hexyl laurate, diisopropyl adipate; and alcohol or polyalcohol heptanoates, octanoates, decanoates or ricinoleates, for example fatty alcohols such as propylene glycol dioctanoate, and isopropyl N-lauroyl sarcosinate (in particular Eldew-205SL from Ajinomoto); hydroxyl esters such as isostearyl lactate, diisostearyl malate; and pentaerythritol esters; branched C8-C16 esters, in particular isohexyl neopentanoate.

2/Plant hydrocarbon oils having a high content of triglycerides constituted of fatty acid esters of glycerol in which the fatty acids may have varied chain lengths from C₄ to C₂₄, it being possible for these chains to be linear or branched, saturated or unsaturated; these oils are in particular wheatgerm oil, corn oil, sunflower oil, shea oil, castor oil, sweet almond oil, macadamia oil, apricot oil, soybean oil, rapeseed oil, cotton seed oil, lucerne oil, poppy seed oil, pumpkin seed oil, sesame oil, gourd oil, avocado oil, hazelnut oil, grape seed or blackcurrant seed oil, evening primrose oil, millet oil, barley oil, quinoa oil, olive oil, rye oil, safflower oil, candlenut oil, passion flower oil, musk rose oil, jojoba oil, palm oil, calophyllum oil; or alternatively triglycerides of caprylic/capric acids such as those sold by the company Stearinerie Dubois or those sold under the names “Miglyol 810®”, “812®” and “818®” by the company Dynamit Nobel.

3/C6-C32, in particular C12-C26, alcohols, and in particular monoalcohols, such as oleyl alcohol, linoleyl alcohol, linolenyl alcohol, isostearyl alcohol, 2-hexyldecanol, 2-butyloctanol, 2-undecylpentadecanol and octyldodecanol.

4/Volatile on non-volatile, linear or branched hydrocarbon oils of synthetic or mineral origin, which may be chosen from hydrocarbon oils having from 5 to 100 carbon atoms, and in particular petroleum jelly, polydecenes, hydrogenated polyisobutenes such as Parleam, squalane, perhydrosqualene and mixtures thereof.

There may be mentioned more particularly linear, branched and/or cyclic C5-C48 alkanes, and preferably branched C8-C16 alkanes such as C8-C16 isoalkanes of petroleum origin (also called isoparaffins); in particular decane, heptane, dodecane, cyclohexane; and isododecane, isodecane, isohexadecane.

5/Volatile or non-volatile silicone oils; as volatile silicone oils, there may be mentioned linear or cyclic volatile silicone oils, in particular those having a viscosity of less than 8 centistokes, and having in particular from 2 to 10 silicon atoms, these silicones optionally containing alkyl or alkoxy groups having from 1 to 22 carbon atoms; and in particular octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, methylhexyldimethylsiloxane and mixtures thereof.

The non-volatile silicone oils which can be used according to the invention may be polydimethylsiloxanes (PDMS), polydimethylsiloxanes containing alkyl or alkoxy groups which are pendent and/or at the silicone chain end, groups each having from 2 to 24 carbon atoms, phenylated silicones such as phenyltrimethicones, phenyldimethicones, phenyltrimethylsiloxydiphenylsiloxanes, diphenyldimethicones, diphenylmethyldiphenyltrisiloxanes, 2-phenylethyl trimethylsiloxysilicates.

Preferably, the physiologically acceptable medium of the composition according to the invention comprises, in a liquid fatty phase, at least one oil and/or solvent chosen from, alone or as a mixture, isododecane, Parleam, isononyl isononanoate, octyldodecanol, phenyl trimethicone, C12-C15 alkyl benzoates and/or D5 (decamethylcyclopentasiloxane).

The liquid fatty phase may additionally comprise additional oils and/or solvents which may be chosen from, alone or as a mixture:
fluorinated oils such as perfluoropolyethers, perfluoroalkanes, such as perfluorodecalin, perfluorodamantanes, monoesters, diesters and triesters of perfluoroalkyl phosphates and fluorinated ester oils;
oils of animal origin;
C₆ to C₄₀, in particular C10-C40, ethers; propylene glycol ethers that are liquid at room temperature, such as propylene glycol monomethyl ether, propylene glycol monomethyl ether acetate, dipropylene glycol mono-n-butyl ether;
C₈-C₃₂ fatty acids, such as oleic acid, linoleic acid, linolenic acid and mixtures thereof;
bifunctional oils, comprising two functional groups chosen from an ester and/or an amide and comprising from 6 to 30 carbon atoms, in particular 8 to 28 carbon atoms, even better from 10 to 24 carbons, and 4 heteroatoms chosen from 0 and N; preferably amide and ester functional groups being in the chain;
ketones that are liquid at room temperature (25° C.) such as methyl ethyl ketone, methyl isobutyl ketone, diisobutyl ketone, isophorone, cyclohexanone, acetone;
aldehydes that are liquid at room temperature, such as benzaldehyde, acetaldehyde.

The liquid fatty phase may represent 1 to 90% by weight of the composition, in particular 5 to 75% by weight, in particular 10 to 60% by weight, or even 25 to 55% by weight, of the total weight of the composition.

The composition according to the invention may advantageously comprise a thickening agent which may be chosen in particular from:
silicas, in particular hydrophobic silicas, such as those described in the document EP-A-898960, and for example marketed under the references “AEROSIL R812®” by the company Degussa, “CAB-O-SIL TS-530®”, “CAB-O-SIL TS-610®”, “CAB-O-SIL TS-720®” by the company Cabot, “AEROSIL R972®”, “AEROSIL R974®” by the company Degussa;
clays such as montmorillonite, modified clays such as bentones for example, stearalkonium hectorite, stearalkonium bentonite,
alkyl ethers of polysaccharides (in particular whose alkyl group contains from 1 to 24 carbon atoms, preferably from 1 to 10, even better from 1 to 6, and more especially from 1 to 3) such as those described in the document EP-A-898958.

The quantity of thickening agent in the composition according to the invention may range from 0.05 to 40% by weight, relative to the total weight of the composition, preferably from 0.5 to 20% and even better from 1 to 15% by weight.

The composition according to the invention may also comprise at least one wax of plant, animal, mineral or synthetic origin, or even silicone wax.

There may be mentioned, in particular, alone or as a mixture, hydrocarbon waxes such as beeswax, Carnauba wax, Candelilla wax, Ouricoury wax, Japan wax, cork fibre wax or sugarcane wax; paraffin wax, lignite wax, microcrystalline waxes; lanolin wax; Montan wax; ozokerites; polyethylene waxes; waxes obtained by Fischer-Tropsch synthesis; hydrogenated oils, fatty esters and glycerides that are concrete at 25° C. It is also possible to use silicone waxes, among which there may be mentioned alkyl polymethylsiloxane, alkoxy polymethylsiloxane and/or polymethylsiloxane esters.

The quantity of wax in the composition according to the invention may range from 0.1 to 70% by weight, relative to the total weight of the composition, preferably from 1 to 40% by weight, and even better from 5 to 30% by weight.

The composition according to the invention may also comprise one or more colouring matters chosen from pulverulent compounds such as pigments, fillers, pearlescent agents and glitter, and/or fat-soluble or water-soluble colorants. The colouring matter, in particular the pulverulent colouring matter, may be present in the composition in an amount of 0.01 to 50% by weight, relative to the weight of the composition, preferably 0.1 to 40% by weight, or even 1 to 30% by weight. The expression pigments should be understood to mean particles of any shape, that are white or coloured, inorganic or organic, insoluble in physiological medium, intended to colour the compositions. The expression pearlescent agents should be understood to mean iridescent particles of any shape, in particular produced by certain molluscs in their shell or else synthesized.

The pigments may be white or coloured, inorganic and/or organic, interferential or otherwise. There may be mentioned, among the inorganic pigments, titanium dioxide, optionally surface-treated, zirconium or cerium oxides, and iron or chromium oxides, manganese violet, ultramarine blue, chromium hydrate and ferric blue. Among the organic pigments, there may be mentioned carbon black, D&C type pigments, and carmine-, barium-, strontium-, calcium and aluminium-based lacquers. The pearlescent pigments may be chosen from white pearlescent pigments such as mica coated with titanium or with bismuth oxychloride, coloured pearlescent pigments such as mica-titanium with iron oxides, mica-titanium with in particular ferric blue or chromium oxide, mica-titanium with an organic pigment of the abovementioned type and bismuth oxychloride-based pearlescent pigments.

The fillers may be inorganic or organic, lamellar or spherical. There may be mentioned talc, mica, silica, kaolin, nylon and polyethylene powders, poly-β-alanine and polyethylene powders, Teflon, lauroyllysine, starch, boron nitride, tetrafluoroethylene polymer powders, hollow microspheres such as Expancel (Nobel Industrie), polytrap (Dow Corning) and microbeads of silicone resin (Tospearls from Toshiba, for example), precipitated calcium carbonate, magnesium carbonate and hydrocarbonate, hydroxyapatite, hollow silica microspheres (SILICA BEADS from MAPRECOS), glass or ceramic microcapsules, metal soaps derived from organic carboxylic acids having from 8 to 22 carbon atoms, preferably from 12 to 18 carbon atoms, for example zinc stearate, magnesium stearate or lithium stearate, zinc laurate, magnesium myristate.

The fat-soluble colorants are for example Sudan red, DC Red 17, DC Green 6, β-carotene, soybean oil, Sudan brown, DC Yellow 11, DC Violet 2, DC Orange 5, quinoline yellow. They may represent 0.01 to 20% of the weight of the composition, and even better from 0.1 to 6%.

The water-soluble colorants are, for example, beet juice, methylene blue, and may represent 0.01 to 6% of the total weight of the composition.

The composition may additionally comprise other ingredients commonly used in cosmetic compositions. Such ingredients may be chosen from antioxidants, perfumes, essential oils, preservatives, cosmetic active agents, moisturizers, vitamins, ceramides, sunscreens, surfactants, spreading agents, wetting agents, dispersing agents, antifoams, neutralizers, stabilizers, polymers and in particular fat-soluble film-forming polymers, and mixtures thereof.

Of course, persons skilled in the art will be careful to choose this or these optional additional compounds, and/or their quantity, such that the advantageous properties of the composition for use according to the invention are not, or not substantially, impaired by the addition envisaged.

The compositions according to the invention may be provided in any acceptable and customary form for a cosmetic or pharmaceutical composition.

They may therefore be provided in the form of a suspension, a dispersion, in particular an oil-in-water dispersion by means of vesicles; an organic or oily solution that is optionally thickened or even gelled; an oil-in-water, water-in-oil or multiple emulsion; a gel or a foam; an oily or emulsified gel; a dispersion of vesicles, in particular lipid vesicles; a two-phase or multiphase lotion; a spray; a lotion, a cream, an ointment, a soft paste, an unguent, a cast or moulded solid, and in particular as a stick or in a dish, or as a compact solid.

Persons skilled in the art will be able to choose the appropriate galenic form, and its method of preparation, on the basis of their general knowledge, taking into account, on the one hand, the nature of the constituents used, in particular their solubility in the carrier, and, on the other hand, the application envisaged for the composition.

The compositions in accordance with the invention have improved gloss and staying power of the said gloss compared with the state of the art, they can be used for caring for or making up keratin materials such as the hair, the skin, the eyelashes, the eyebrows, the nails, the lips, the scalp and more particularly for making up the lips, the eyelashes and/or the face.

They may therefore be provided in the form of a care and/or make-up product for the skin of the body or of the face, for the lips, the eyelashes, the eyebrows, the hair, the scalp or the nails; an anti-sun or a self-tanning product; a hair product, in particular for dyeing, conditioning and/or caring for the hair; they are advantageously provided in the form of a mascara, a lipstick, a lip gloss, a blusher or an eyeshadow, or a foundation.

The subject of the invention is also a method for the cosmetic treatment of keratin materials, in particular the skin of the body or of the face, the lips, the nails, the hair and/or the eyelashes, comprising the application to the said materials of a cosmetic composition as defined above.

This method according to the invention makes it possible in particular to care for or make up the lips by applying a lipstick or lip gloss composition according to the invention.

The invention is illustrated in greater detail by the following examples.

### EXAMPLE 1

### Synthesis of Pentaerythrityl Isophthalate/Isostearate

280 g of isostearic acid and 100 g of pentaerythritol are loaded into a reactor equipped with mechanical stirring, an argon inlet and a distillation system, and then the mixture is gradually heated, under a gentle argon stream, to 110-130° C. in order to obtain a homogeneous solution. The temperature is then gradually increased to 180° C. and it is maintained for about 2 hours. The temperature is again increased to 220° C. and it is maintained until an acid value of less than or equal to 1 is obtained, which takes about 11 hours. The mixture is cooled to a temperature of between 100 and 130° C. and then 100 g of isophthalic acid are introduced and the mixture is again gradually heated to 220° C. for about 11 hours.

420 g of pentaerythrityl isophthalate/isostearate polycondensate are thus obtained in the form of a very thick oil.

The polycondensate has the following characteristics:
acid value=3.5
η110° C.=2280 mPa·s

### EXAMPLE 2

### Synthesis of Diglyceryl Sebacate/Isostearate

295 g of isostearic acid, 100 g of diglycerol and 105 g of sebacic acid are loaded into a reactor equipped with mechanical stirring, an argon inlet and a distillation system, and then the mixture is gradually heated to 220° C. and the temperature is maintained for about 7 hours.

430 g of polycondensate are thus obtained in the form of a very thick oil.

The polycondensate has the following characteristics:
acid value=5.3
η110° C.=1040 mPa·s

### EXAMPLE 3

### Synthesis of Glyceryl Sebacate/Isostearate

280 g of isostearic acid, 85 g of glycerol and 135 g of sebacic acid are loaded into a reactor equipped with mechanical stirring, an argon inlet and a distillation system, and then the mixture is gradually heated to 220° C. and the temperature is maintained for about 7 hours.

410 g of polycondensate are thus obtained in the form of a thick oil.

The polycondensate has the following characteristics:
acid value=6.4
μ80° C.=164 mPa·s

### EXAMPLE 4

### Synthesis of Sorbitol Sebacate/Isostearate

305 g of isostearic acid, 105 g of sorbitol and 90 g of sebacic acid are loaded into a reactor equipped with mechanical stirring, an argon inlet and a distillation system, and then the mixture is gradually heated to 180° C. and the temperature is maintained for about 30 hours.

400 g of polycondensate are thus obtained in the form of a very thick oil.

The polycondensate has the following characteristics:
acid value=20.8
μ80° C.=584 mPa·s

### EXAMPLE 5

A lipstick having the following composition was prepared:

| | | |
|---|---|---|
| Polycondensate of Example 1 | 30 | g |
| Polyethylene wax | 11 | g |
| Pigments and fillers | 7 | g |
| Parleam (hydrogenated isoparaffin) | qs 100 | g |

A coloured and glossy film is obtained, after application to the lips, which remains glossy for at least 2 hours.

It is possible to prepare a lipstick in a similar manner by replacing the polycondensate of Example 1 with that of Example 2, 3 or 4.

### EXAMPLE 6

### Synthesis of Diglyceryl Isostearate/Glutamate/Sebacate

280 g of isostearic acid, 115 g of diglycerol, 15 g of glutamatic acid and 90 g of sebacic acid are loaded into a reactor equipped with mechanical stirring, an argon inlet and a distillation system, and then the mixture is heated to 230° C. and the temperature is maintained for about 5 hours.

430 g of polycondensate are thus obtained in the form of a thick oil.

The polycondensate has the following characteristics:
acid value=8.0
η80° C.=300 mPa·s

### EXAMPLE 7

### Synthesis of Glyceryl Sebacate/Transaconitate/Isostearate

270 g of isostearic acid, 90 g of diglycerol, 35 g of trans-aconitic acid and 105 g of sebacic acid are loaded into a reactor equipped with mechanical stirring, an argon inlet and a distillation system, and then the mixture is gradually heated to 230° C. and the temperature is maintained for about 8 hours.

440 g of polycondensate are thus obtained in the form of a thick oil.

The polycondensate has the following characteristics:
acid value=1.2
η80° C.=228 mPa·s

### EXAMPLE 8

### Synthesis of Diglyceryl Sebacate/Transaconitate/Isostearate

295 g of isostearic acid, 105 g of diglycerol, 15 g of trans-aconitic acid and 85 g of sebacic acid are loaded into a reactor equipped with mechanical stirring, an argon inlet and a distillation system, and then the mixture is gradually heated to 220° C. and the temperature is maintained for about 7 hours.

450 g of polycondensate are thus obtained in the form of a thick oil.

The polycondensate has the following characteristics:
acid value=4.2
η80° C.=384 mPa·s

### EXAMPLE 9

### Synthesis of Diglyceryl Sebacate/Transaconitate/Itaconate/Isostearate

280 g of isostearic acid, 115 g of diglycerol, 15 g of trans-aconitic acid, 10 g of itaconic anhydride and 80 g of sebacic acid are loaded into a reactor equipped with mechanical stirring, an argon inlet and a distillation system, and then the mixture is gradually heated to 220° C. and the temperature is maintained for about 8 hours.

450 g of polycondensate are thus obtained in the form of a thick oil.

The polycondensate has the following characteristics:
acid value=4.5
η180° C.=555 mPa·s

1. A composition comprising, in a cosmetically or pharmaceutically acceptable medium, at least one polycondensate capable of being obtained by a process comprising reacting the following monomers:
from 10 to 30% by weight, relative to the total weight of the polycondensate, of at least one polyol comprising 3 to 6 hydroxyl groups;
from 30 to 80% by weight, relative to the total weight of the polycondensate, of at least one linear, branched or cyclic, saturated or unsaturated, non-aromatic monocarboxylic acid comprising 6 to 32 carbon atoms;
from 1 to 40% by weight, relative to the total weight of the polycondensate, of at least one polycarboxylic acids or cyclic anhydrides of such a polycarboxylic acid or a lactone comprising at least one COOH group;
0.1 to 15% by weight, relative to the total weight of the polycondensate, of at least one silicone having a hydroxyl or carboxyl functional group, or mixtures thereof.

2. The composition according to claim 1, wherein the at least one polyol is at least one selected from the group consisting of:
a triol, 1,2,4-butanetriol, 1,2,6-hexanetriol, trimethylolethane, trimethylolpropane, glycerol,
a tetraols, pentaerythritol, erythritol, diglycerol, ditrimethylolpropane,
a pentol such as xylitol,
a hexol such as sorbitol, mannitol, dipentaerythritol and triglycerol.

3. The composition according to claim 1, wherein the polyol at least one represents 12 to 25% by weight of the total weight of the polycondensate.

4. The composition according to claim 1, wherein the non-aromatic monocarboxylic acid is at least one saturated monocarboxylic acid selected from the group consisting of:
caproic acid, caprylic acid, isoheptanoic acid, 4-ethylpentanoic acid, 2-ethylhexanoic acid, 4,5-dimethylhexanoic acid, 2-heptylheptanoic acid, 3,5,5-trimethyl-hexanoic acid, octanoic acid, isooctanoic acid, nonanoic acid, decanoic acid, isononanoic acid, lauric acid, tridecanoic acid, myristic acid, palmitic acid, stearic acid, isostearic acid, arachidic acid, behenic acid, cerotic (hexacosanoic) acid; cyclopentanecarboxylic acid, cyclopentaneacetic acid, 3-cyclopentylpropionic acid, cyclohexanecarboxylic acid, cyclohexylacetic acid, and 4-cyclohexylbutyric acid
unsaturated but non-aromatic monocarboxylic acids consisting of caproleic acid, obtusilic acid, undecylenic acid, dodecylenic acid, linderic acid, myristoleic acid, physeteric acid, tsuzuic acid, palmitoleic acid, oleic acid, petroselinic acid, vaccenic acid, elaidic acid, gondoic acid, gadoleic acid, erucic acid, cetoleic acid, nervonic acid, linoleic acid, linolenic acid, and arachidonic acid.

5. The composition according to claim 1, wherein the at least one non-aromatic monocarboxylic acid, represents 40 to 75% by weight of the total weight of the final polycondensate.

6. The composition according to claim 1, wherein the polycarboxylic acid or its anhydride, is
a saturated or unsaturated, or even aromatic, linear, branched or cyclic polycarboxylic acid comprising 2 to 50 carbon atoms; said acid comprising at least two carboxyl groups COOH; and optionally capable of comprising 1 to 10 heteroatoms, which are identical or different, chosen from O, N and S; or optionally capable of comprising at least one perfluorinated radical chosen from —CF₂— (divalent) or —CF₃; and its anhydride or mixtures thereof; or
a polycarboxylic acid having a linear or branched, saturated or unsaturated chain comprising at least one heteroatom chosen from O, N or S, in particular 1 to 10 heteroatoms, which are identical or different, or comprising at least one perfluorinated radical —CF₂— or —CF₃ and having moreover at least 2 carboxyl groups COOH; and its anhydride or mixtures thereof; or,
a saturated or unsaturated, or even aromatic, heterocyclic polycarboxylic acid comprising at least one heteroatom chosen from O, N or S, in particular 1 to 10 heteroatoms, which are identical or different, and at least 2 carboxyl groups COOH; and its anhydride or mixtures thereof; or,
a polycarboxylic acid derived from a sugar, comprising at least 2 carboxyl groups COOH; and its anhydride, or mixtures thereof;
itaconic anhydride and the 1,4-monoanhydride of 1,4,5,8-naphthalenetetracarboxylic acid;
polycarboxylic (including heterocyclic) amino acid, that is to say polycarboxylic acids having a linear, branched or cyclic, saturated or unsaturated chain optionally comprising at least one heteroatom chosen from O, N or S, in particular 1 to 10 heteroatoms which are identical or different, or optionally comprising at least one perfluorinated radical —CF₂— or —CF₃; and additionally comprising at least one primary, secondary or tertiary amine functional group and having moreover at least 2 carboxyl groups COOH; and its anhydride, or mixtures thereof.

7. The composition according to claim 1, wherein the polycarboxylic acid, or its anhydride, is at least one selected from the group consisting of;
decanedioic acid, dodecanedioic acid, cyclopropanedicarboxylic acid, cyclohexanedicarboxylic acid, cyclobutanedicarboxylic acid, 1,4-naphthalenedicarboxylic acid, 2,3-naphthalenedicarboxylic acid, 2,6-naphthalenedicarboxylic acid, suberic acid, oxalic acid, malonic acid, succinic acid, phthalic acid, terephthalic acid, isophthalic acid, tetrahydrophthalic acid, hexahydrophthalic acid, pimelic acid, sebacic acid, azelaic acid, glutaric acid, adipic acid, fumaric acid, maleic acid, itaconic acid, dimers of fatty acids;
cyclohexanetricarboxylic acid, trimellitic acid, 1,2,3-benzenetricarboxylic acid, 1,3,5-benzenetricarboxylic acid; aconitic acid
butanetetracarboxylic acid and pyromellitic acid,
2,2′-[1,5-pentanediylbis(thio)]bis-acetic acid
6,6′-[(1,2-dioxo-1,2-ethanediyl)diimino]bis-hexanoic acid
2,2′-sulphinylbis-acetic acid
4,13-dioxo-3,5,12,14-tetraazahexadecanedioic acid
poly(ethylene glycol)disuccinate, in particular having a mass of 250-600
poly(ethylene glycol)bis(carboxymethyl)ether, in particular having a mass of 250-600
poly[oxy(1,2-dicarboxy-1,2-ethanediyl)], in particular having a DP<10
8-[(carboxymethyl)amino]-8-oxooctanoic acid
2,2′-[methylenebis(sulphonyl)]bis-acetic acid
4,4′-(1,6-hexanediyldiimino)bis[4-oxo-butanoic] acid
4,9-dioxo-3,5,8,10-tetraazadodecanedioic acid
4-[(1-carboxyethyl)amino]-4-oxo-butanoic acid
6-[(3-carboxy-1-oxopropyl)amino]hexanoic acid
N,N′-(1,6-dioxo-1,6-hexanediyl)bis-glycine
N,N′-(1,6-dioxo-1,6-hexanediyl)bis-phenylalanine
N,N′-(1,3-dioxo-1,3-propanediyl)bis-glycine
4,4′-[(1,4-dioxo-1,4-butanediyl)diimino]bis-butanoic acid
4,4′-[(1,6-dioxo-1,6-hexanediyl)diimino]bis-butanoic acid
6,6′-[1,6-hexanediylbis(iminocarbonylimino)]bis-hexanoic acid
N-benzoyl-S-(carboxymethyl)-cysteine
N,N′-(2,2,3,3-tetrafluoro-1,4-dioxo-1,4-butanediyl)bis-glycine
N,N′-(2,2,3,3-tetrafluoro-1,4-dioxo-1,4-butanediyl)bis-alanine
4,4′-[(2,2,3,3-tetrafluoro-1,4-dioxo-1,4-butanoic acid
N,N′-(1,5-dioxo-1,5-pentanediyl)bis-glycine
N,N′-(1,9-dioxo-1,9-nonanediyl)bis-glycine
N,N′-(1,10-dioxo-1,10-decanediyl)bis[N-methyl-glycine
bis(3-carboxypropyl)ester of propanedioic acid
7,16-dioxo-6,8,15,17-tetraazadocosanedioic acid
N-benzoyl-N-(2-carboxyethyl)-glycine
[2-[(2-carboxymethyl)amino]-2-oxoethyl]-benzenepropanoic acid
[2-[(2-carboxyethyl)amino]-2-oxoethyl]-benzenepropanoic acid
4,7,9,12-tetraoxapentadecanedioic acid
2,3-pyridinedicarboxylic acid
4-pyranone-2,6-dicarboxylic acid
2,5-pyrazinedicarboxylic acid
2,5-pyridinedicarboxylic acid
2,3-benzofurandicarboxylic acid
7-oxabicyclo[2.2.1]heptane-2,3-dicarboxylic acid
3,4-pyridinedicarboxylic acid
2,4-pyridinedicarboxylic acid
3,5-pyridinedicarboxylic acid
2,6-pyridinedicarboxylic acid
1H-imidazole-4,5-dicarboxylic acid
2,3-quinolinedicarboxylic acid
6,6,7,7-tetrafluoro-3-oxabicyclo[3.2.0]heptane-2,4-dicarboxylic acid
2,6-pyrazinedicarboxylic acid
2,6-dimethyl-3,5-pyridinedicarboxylic acid
1-phenyl-1H-pyrazole-3,4-dicarboxylic acid
2,5-furandicarboxylic acid
3,4-furandicarboxylic acid
1,2,5-thiadiazole-3,4-dicarboxylic acid
1,4-dihydro-1,2,4,5-tetrazine-3,6-dicarboxylic acid
2,3-furandicarboxylic acid
3,4-thiophenedicarboxylic acid
1H-1,2,3-triazole-4,5-dicarboxylic acid
2-methylimidazole-4,5-dicarboxylic acid
2,4-quinolinedicarboxylic acid
naphtho[2,1-b]furan-1,2-dicarboxylic acid
3,4-quinolinedicarboxylic acid
7-oxabicyclo[2.2.1]hept-5-ene-2,3-dicarboxylic acid
2,3-quinoxalinedicarboxylic acid
1,4-piperazinedicarboxylic acid
2,5-dimethyl-3,4-furandicarboxylic acid
tetrahydro-2,5-thiophenedicarboxylic acid
4-phenyl-3,5-pyridinedicarboxylic acid
thieno[3,2-b]thiophene-2,5-dicarboxylic acid
3-methyl-2,4-thiophenedicarboxylic acid
naphthostyril-5,6-dicarboxylic acid
3-phenyl-2,4-quinolinedicarboxylic acid
3,4-dimethyl-2,5-dicarboxythiophene
3,4-diphenyl-2,5-thiophenedicarboxylic acid
2,5-diphenyl-3,4-furandicarboxylic acid
7-oxo-7H-benzimidazo[2,1-a]benz[de]isoquinoline-3,4-dicarboxylic acid
2,3-dihydro-1,3-dioxo-1H-benz[de]isoquinoline-6,7-dicarboxylic acid
3,4-bis(phenylmethoxy)-2,5-furandicarboxylic acid
4,4′-bibenzoic acid 2,2′-sulphone
2,7-diphenyl-m-anthrazoline-4,5-dicarboxylic acid
2,4-pyrimidinedicarboxylic acid
2-phenyl-4,5-thiazoledicarboxylic acid
6-phenyl-2,3-pyridinedicarboxylic acid
5,6-dimethyl-2,3-pyrazinedicarboxylic acid
3,7-dibenzothiophenedicarboxylic acid
9-oxo-9H-xanthene-1,7-dicarboxylic acid
2-(1,1-dimethylethyl)-H-imidazole-4,5-dicarboxylic acid
6,7-quinolinedicarboxylic acid
6-methyl-2,3-pyridinedicarboxylic acid
4,5-pyrimidinedicarboxylic acid
2-methyl-3,4-furandicarboxylic acid
1,2-indolizinedicarboxylic acid
2,8-dibenzothiophenedicarboxylic acid
3,6-pyridazinedicarboxylic acid
1,10-phenanthroline-2,9-dicarboxylic acid
1,4,5,6-tetrahydro-5,6-dioxo-2,3-pyrazinedicarboxylic acid
3,4-dimethoxy-2,5-furandicarboxylic acid
2-ethyl-4,5-imidazoledicarboxylic acid
2-propyl-1H-imidazole-4,5-dicarboxylic acid
4-phenyl-2,5-pyridinedicarboxylic acid
4,5-pyridazinedicarboxylic acid
1,4,5,8-tetrahydro-1,4:5,8-diepoxynaphthalene-4a,8a-dicarboxylic acid
5,5-dioxide-2,8-dibenzothiophenedicarboxylic acid
pyrazolo[1,5-a]pyridine-2,3-dicarboxylic acid
2,3-dihydro-1H-pyrrolizine-1,7-dicarboxylic acid
6-methyl-2,4,5-pyridinetricarboxylic acid
pyrrolo[2,1,5-cd]indolizine-5,6-dicarboxylic acid
3,4-bis(2,2,3,3,4,4,4-heptafluorobutyl)-1H-pyrrole-2,5-dicarboxylic acid
6,7,9,10,17,18,20,21-octahydrodibenzo[b,k][1,4,7,10,13,16]hexaoxacyclooctadecine-2,14-dicarboxylic acid
6,7,9,10,17,18,20,21-octahydrodibenzo[b,k][1,4,7,10,13,16]hexaoxacyclooctadecine-2,13-dicarboxylic acid
2-methyl-3,4-quinolinedicarboxylic acid
4,7-quinolinedicarboxylic acid
3,5-isoxazoledicarboxylic acid
2-(trifluoromethyl)-3,4-furandicarboxylic acid
5-(trifluoromethyl)-2,4-furandicarboxylic acid
6-methyl-2,4-quinolinedicarboxylic acid
5-oxo-1,2-pyrrolidinedicarboxylic acid
5-ethyl-2,3-pyridinedicarboxylic acid
1,2-dihydro-2-oxo-3,4-quinolinedicarboxylic acid
4,6-phenoxathiindicarboxylic acid
10,10-dioxide 1,9-phenoxathiindicarboxylic acid
3,4-dihydro-2H-1,4-thiazine-3,5-dicarboxylic acid
2,7-di(tert-butyl)-9,9-dimethyl-4,5-xanthenedicarboxylic acid
6-methyl-2,3-quinoxalinedicarboxylic acid
3,7-quinolinedicarboxylic acid
2,5-quinolinedicarboxylic acid
2-methyl-6-phenyl-3,4-pyridinedicarboxylic acid
3,4-dimethylthieno[2,3-b]thiophene-2,5-dicarboxylic acid
3,4-dimethoxythiophene-2,5-dicarboxylic acid
5-methyl-3,4-isoxazoledicarboxylic acid
2,6-bis(aminocarbonyl)-3,5-pyridinedicarboxylic acid
3,5-bis(aminocarbonyl)-2,6-pyrazinedicarboxylic acid
2,3-pyridinedicarboxylic acid
6-(1,1-dimethylethyl)-2-ethyl-3,4-pyridinedicarboxylic acid
3-methyl-5-phenyl-2,4-thiophenedicarboxylic acid
1,2-dihydro-2-oxo-6-phenyl-3,5-pyridinedicarboxylic acid
8-methyl-2,4-quinolinedicarboxylic acid
4-ethyl-2,6-dimethyl-3,5-pyridinedicarboxylic acid
5-(phenoxymethyl)-2,4-furandicarboxylic acid
5-(acetylamino)-3-methyl-2,4-thiophenedicarboxylic acid
2-(4-heptylphenyl)-4,8-quinolinedicarboxylic acid
2,8-bis(4-heptylphenyl)pyrido[3,2-g]quinoline-4,6-dicarboxylic acid
1,2,3,4,6,7,8,9-octahydro-2,8-dioxopyrido[3,2]quinoline-3,7-dicarboxylic acid
2,8-dimethylpyrido[3,2-g]quinoline-3,7-dicarboxylic acid
5,6-quinolinedicarboxylic acid
6-ethyl-2-methylcinchomeronic acid
2-methyl-6-propylcinchomeronic acid
6-isopropyl-2-methylcinchomeronic acid
6-tert-butyl-2-methylcinchomeronic acid
1,4-dimethyl-7-oxabicyclo[2.2.1]heptane-2,3-dicarboxylic acid
1,2-dihydro-2-oxo-3,8-quinolinedicarboxylic acid
1,2-dihydro-2-oxo-3,6-quinolinedicarboxylic acid
1,2-dihydro-2-oxo-3,7-quinolinedicarboxylic acid
3,7-dimethyl-2,8-diphenylpyrido[3,2-g]quinoline-4,6-dicarboxylic acid
8-methyl-2,3-quinolinedicarboxylic acid
3-[[(1,1-dimethylethyl)amino]sulphonyl]-2,5-thiophenedicarboxylic acid
4-(acetylamino)-2,3-thiophenedicarboxylic acid
2,5-pyridinedicarboxylic acid
2,6-pyridinedicarboxylic acid
2,4-thiophenedicarboxylic acid
2,5-thiophenedicarboxylic acid
1,4-pyran-2,6-dicarboxylic acid
ribaric acid
glucaric acid
xylaric acid
arabinaric acid
mannaric acid
idaric acid
altraric acid
L-glucaric acid
L-arabinaric acid
allaric acid
galactaric acid
meso-tartaric acid
D-glucaric acid
L-idaric acid
hexaric acid
2,3-dihydroxybutanedioic acid
D-tartaric acid
DL-tartaric acid
D-glucaric acid
tartaric acid
tetrahydroxysuccinic acid
2-carboxy-2,3-dideoxy-D-manno-2-octulopyranosonic acid
methyl 3-deoxy-D-arabino-2-heptulopyranosaric acid
D-lyxo-2-heptulopyranosaric acid
2,6-anhydro-L-glycero-L-galactoheptaric acid
1,4-dihydro-4-oxo-2,6-pyridinedicarboxylic acid
2,6-piperidinedicarboxylic acid
1H-pyrrole-3,4-dicarboxylic acid
4-amino-2,6-dicarboxylic acid
1-methyl-1H-pyrazole-3,4-dicarboxylic acid
2,3-piperidinedicarboxylic acid
1-methyl-1H-imidazole-4,5-dicarboxylic acid
2,4-thiazolidinedicarboxylic acid
1-(phenylmethyl)-1H-imidazole-4,5-dicarboxylic acid
5-amino-6-oxo-2,3-piperidinedicarboxylic acid
5-amino-6-oxo-2,4-piperidinedicarboxylic acid
5-amino-6-oxo-[2S-(2α,4β,5α)]-2,4-piperidinedicarboxylic acid
(2S,4R)-2,4-pyrrolidinedicarboxylic acid
(2S-cis)-2,4-pyrrolidinedicarboxylic acid
2-amino-1H-imidazole-4,5-dicarboxylic acid
2,5-pyrrolidinedicarboxylic acid
4-amino-3,5-isothiazoledicarboxylic acid
1-methyl-1H-pyrazole-3,5-dicarboxylic acid
7-(diethylamino)-2-oxo-2H-1-benzopyran-3,4-dicarboxylic acid
3,4-diethyl-1H-pyrrole-2,5-dicarboxylic acid
1-phenyl-1H-pyrrole-3,4-dicarboxylic acid
cis-2,3-piperazinedicarboxylic acid
2,3-piperazinedicarboxylic acid
2,5-piperazinedicarboxylic acid
2,6-piperazinedicarboxylic acid
2-amino-3,5-pyridinedicarboxylic acid
2-methylpyrrole-3,4-dicarboxylic acid
4-(methylamino)-2,6-pyridinedicarboxylic acid
2-amino-6-methyl-3,4-pyridinedicarboxylic acid
5-amino-2-methyl-3,4-pyridinedicarboxylic acid
2-amino-6-methyl-3,5-pyridinedicarboxylic acid
2,5-dimethylpyrrole-3,4-dicarboxylic acid
2-amino-6-hydroxy-3,5-pyridinedicarboxylic acid
2,4-pyrrolidinedicarboxylic acid
1H-indole-2,4-dicarboxylic acid
1H-indole-2,6-dicarboxylic acid
1H-indole-2,5-dicarboxylic acid
5-phenyl-2,4-pyrrolidinedicarboxylic acid
5-methyl-2,4-pyrrolidinedicarboxylic acid
trans-2,4-azetidinedicarboxylic acid
cis-2,4-azetidinedicarboxylic acid
3,5-piperidinedicarboxylic acid
2,3-pyrrolidinedicarboxylic acid
2,3-azetidinedicarboxylic acid
3,4-pyrrolidinedicarboxylic acid
2,3-dihydro-6H-1,4-dioxino[2,3-c]pyrrole-5,7-dicarboxylic acid
1H-imidazole-2,4-dicarboxylic acid
1-butyl-1H-pyrrole-2,3-dicarboxylic acid
3-amino-1-oxide-2,4-pyridinedicarboxylic acid
2,3-dihydro-5-phenyl-1H-pyrrolizine-6,7-dicarboxylic acid
3a,4,5,9b-tetrahydro-3H-cyclopenta[c]quinoline-4,6-dicarboxylic acid
3a,4,5,9b-tetrahydro-3H-cyclopenta[c]quinoline-4,8-dicarboxylic acid
2,3-dihydro-1H-imidazole-4,5-dicarboxylic acid
5-amino-6-methyllutidinic acid
1H-indole-3,7-dicarboxylic acid
3,3-dimethyl-2,6-piperidinedicarboxylic acid
1-butyl-2,5-pyrrolidinedicarboxylic acid
1H-indole-4,6-dicarboxylic acid
1-(phenylmethyl)-3,4-pyrrolidinedicarboxylic acid
3-(carboxymethyl)-1H-indole-2,6-dicarboxylic acid
3,4-bis(2,2,2-trifluoroethyl)-1H-pyrrole-2,5-dicarboxylic acid
9-hexyl-9H-carbazole-3,6-dicarboxylic acid
3-methyl-5-(1-piperazinylsulphonyl)-2,4-thiophenedicarboxylic acid
2,3,4,9-tetrahydro-1H-carbazole-5,7-dicarboxylic acid
2,3-dimethyl-1H-indole-4,6-dicarboxylic acid
7-amino-1,4-dihydro-4-oxo-3,6-quinolinedicarboxylic acid
5-amino-3-methyl-2,4-thiophenedicarboxylic acid
(m-tolylimino)diacetic acid
(o-tolylimino)diacetic acid
D-cystathionine
phenethyliminodiacetic acid
2-benzyl-2,2′-iminodiacetic acid
L-α-glutamyl-L-alanyl-L-alanine
N,N′-dibenzylethylenediaminediacetic acid
N-L-γ-glutamyl-D-alanine
glycyl-L-glutamylglycine
N-(carboxymethyl)-N-(tetrahydro-1,1-dioxido-3-thienyl)glycine
N-(2-carboxyethyl)-N-phenyl-β-alanine
N-(carboxymethyl)-N-octylglycine
N-(tert-butoxycarbonyl)iminodiacetic acid
N-(carboxymethyl)-L-alanine
N-(6-aminohexyl)-N-(carboxymethyl)glycine
N-(carboxymethyl)-N-tetradecylglycine
N-(1-carboxyethyl)-D-alanine
N-(carboxymethyl)-D-alanine
decyliminodiacetic acid
3,3′-(dimethylhydrazono)bispropanoic acid
N-(carboxymethyl)-N-[2-(2,6-dioxo-4-morpholinyl)ethyl]glycine
N-α-aspartylglycine
N-β-aspartylglycine
N-L-α-aspartyl-β-alanine
3,4-xylylamino-N,N-diacetic acid
N-(1-carboxyethyl)alanine
N-(carboxymethyl)alanine
N,N′-methylenebisglycine
N-(aminomethyl)-N-(carboxymethyl)glycine
2,2′-(methylhydrazono)bisacetic acid
N-(2-carboxyethyl)-N-(4-methylphenyl)-β-alanine
N-(2-carboxyethyl)-N-(3-methylphenyl)-β-alanine
3-[(carboxymethyl)amino]alanine
D-α-aspartyl-D-alanine
N-(2-carboxyethyl)-N-(1-oxohexadecyl)-β-alanine
N-(2-carboxyethyl)-N-(1-oxodecyl)-β-alanine
N-(2-carboxyethyl)-N-(1-oxotetradecyl)-β-alanine
amino[(carboxymethyl)thio]acetic acid
N,N′-1,6-hexanediylbis-β-alanine
N-(carboxymethyl)-N-phenyl-β-alanine
N-(1-carboxyethyl)-L-alanine
L-glutamic acid
L-aspartic acid
3,3′,3″-[1,2,3-propanetriyltris(oxy)]tris-propanoic acid
pyrazinetricarboxylic acid
4-(3-carboxyphenyl)-2,5-pyridinedicarboxylic acid
3-(carboxymethyl)-2,4-quinolinedicarboxylic acid
3-(carboxymethyl)-1H-indole-2,5-dicarboxylic acid
3-C-carboxy-2-deoxy-D-threopentaric acid
hydroxycitric acid
D-glucopyranuronosyl-D-arabino-2-hexulofuranosidaric acid
2,3,5,6-pyridinetetracarboxylic acid
N,N′-1,2-ethanediylbis[N-(carboxymethyl)-β-alanine
L-α-aspartyl-L-aspartic acid
4-[bis(carboxymethyl)amino]benzoic acid
7-[bis(carboxymethyl)amino]heptanoic acid
N-(2-carboxyethyl)aspartic acid
3-[bis(2-carboxyethyl)amino]benzoic acid
4-[bis(2-carboxyethyl)amino]benzoic acid;
and a cyclic anhydride thereof.

8. The composition according to claim 1, wherein the lactone is at least one lactone selected from the group consisting of:
tetrahydro-2,2-dimethyl-5-oxo-3-furancarboxylic acid
4,7,7-trimethyl-3-oxo-2-oxabicyclo[2.2.1]heptane-1-carboxylic acid
4,6-dimethyl-2-oxo-2H-pyran-5-carboxylic acid
2-oxo-2H-pyran-5-carboxy-2-pentenedioic acid
2-oxo-2H-1-benzopyran-3-carboxylic acid
2-oxo-2H-pyran-6-carboxylic acid
1,3-dihydro-3-oxo-1-isobenzofurancarboxylic acid
4-methyl-2-oxo-2H-1-benzopyran-3-carboxylic acid
1 -oxo-1H-2-benzopyran-3-carboxylic acid
8-methoxy-2-oxo-2H-1-benzopyran-3-carboxylic acid
2-oxo-1-oxaspiro[4.5]decane-4-carboxylic acid
2-oxo-2H-pyran-3-carboxylic acid
4-methyl-2-oxo-2H-pyran-6-carboxylic acid
3-oxo-3H-naphtho[2,1-b]pyran-2-carboxylic acid
tetrahydro-5-oxo-2,3-furandicarboxylic acid
1,3-dihydro-3-oxo-4-isobenzofurancarboxylic acid
1,3-dihydro-1-oxo-5-isobenzofurancarboxylic acid
hexahydro-2-oxo-3,5-methano-2H-cyclopenta[b]furan-7-carboxylic acid
6-methyl-2,4-dioxo-2H-pyran-5-carboxylic acid
1-oxo-3-isochromancarboxylic acid
2-oxo-2H-1-benzopyran-6-carboxylic acid
6-methyl-2-oxo-2H-1-benzopyran-3-carboxylic acid
2,5-dihydro-4,5,5-trimethyl-2-oxo-3-furancarboxylic acid
tetrahydro-5-oxo-2-phenyl-3-furancarboxylic acid
tetrahydro-5-oxo-4-propyl-2-furoic acid
2-butyl-2,3-dideoxypentaric acid
2-oxo-2H-1-benzopyran-7-carboxylic acid
2-oxo-1-oxaspiro[4.4]nonane-4-carboxylic acid
4-ethyltetrahydro-5-oxo-2-furoic acid
5-ethyltetrahydro-2,3-dimethyl-6-oxo-2H-pyran-2-carboxylic acid
7-methoxy-2-oxo-2H-1-benzopyran-3-carboxylic acid
2-oxo-2H-1-benzopyran-4-carboxylic acid
2-oxo-6-pentyl-2H-pyran-3-carboxylic acid
7-oxo-4-oxepanecarboxylic acid
3-(carboxymethyl)-2,3-dideoxypentaric acid
2,3-dihydro-2-oxo-7-benzofurancarboxylic acid
1,3,4,5-tetrahydro-1-oxo-2-benzoxepin-7-carboxylic acid
3,4-dihydro-3-oxo-1H-2-benzopyran-6-carboxylic acid
2,3,4,5-tetrahydro-2-oxo-1-benzoxepin-7-carboxylic acid
3,4-dihydro-1-oxo-1H-2-benzopyran-8-carboxylic acid
1,3,4,5-tetrahydro-3-oxo-2-benzoxepin-9-carboxylic acid
1,3,4,5-tetrahydro-3-oxo-2-benzoxepin-7-carboxylic acid
3,4-dihydro-2-oxo-2H-1-benzopyran-8-carboxylic acid
1,3,4,5-tetrahydro-1-oxo-2-benzoxepin-9-carboxylic acid
3,4-dihydro-1-oxo-1H-2-benzopyran-6-carboxylic acid
3,4-dihydro-3-oxo-1H-2-benzopyran-8-carboxylic acid
2,3,4,5-tetrahydro-2-oxo-1-benzoxepin-9-carboxylic acid
isocitric acid lactone and
5-oxo-2-tetrahydrofurancarboxylic acid.

9. The composition according to claim 1, wherein the polycarboxylic acid or its cyclic anhydride or the lactone, or mixtures thereof, represents 5 to 30% by weight of the total weight of the final polycondensate.

10. The composition according to claim 1, wherein the silicone is of formula: 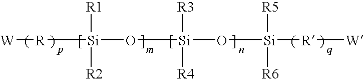
in which:
W and W′ are, independently of each other, OH or COOH;
p and q are, independently of each other, equal to 0 or 1,
R and R′ are, independently of each other, a saturated or unsaturated, or even aromatic, linear, branched or cyclic divalent carbon, in particular hydrocarbon, radical comprising 1 to 12 carbon atoms, and optionally additionally comprising one or more heteroatoms chosen from O, S and N, or mixtures thereof;
R1 to R6 are, independently of each other, a saturated or unsaturated or even aromatic linear, branched or cyclic carbon radical comprising 1 to 20 carbon atoms, or mixtures thereof;
m and n are, independently of each other, integers between 1 and 140, and are such that the weight-average molecular mass (Mw) of the silicone is between 300 and 20 000.

11. The composition according to claim 1, in which the silicone represents 1 to 10% by weight of the total weight of the polycondensate.

12. The composition according to claim 1, in which the polycondensate has at least one of the following characteristics:
an acid value, expressed in mg of potassium hydroxide per g of polycondensate, greater than or equal to 1;
a hydroxyl value, expressed in mg of potassium hydroxide per g of polycondensate, greater than or equal to 40; or
a weight-average molecular mass (Mw) between 1500 and 300 000.

13. The composition according to claim 1, in which the polycondensate has a viscosity such that at least one of the following is satisfied;
at 25° C., when this viscosity can be measured, it is strictly greater than 5000 mPa·s or
the viscosity, measured at 110° C., is between 100 and 4000 mPa·s or
the viscosity, measured at 80° C., is between 150 and 5000 mPa·s.

14. The composition according to claim 1, wherein the polycondensate exhibits solubility in at least one solvent selected from isododecane, Parleam, isononyl isononanoate, octyldodecanol and C12-C15 alkyl benzoate, in an amount of at least 50% by weight, at 70° C.

15. The composition according to claim 1, in which the polycondensate is present in a quantity of between 0.1 and 70% by weight relative to the weight of the final cosmetic or pharmaceutical composition.

16. The composition according to claim 1, wherein the cosmetically or pharmaceutically acceptable medium comprises at least one liquid fatty phase, which may comprise volatile or non-volatile, carbonaceous, hydrocarbon, fluorinated or silicone oils, or mixtures thereof, solvents of inorganic, animal, plant or synthetic origin, or mixtures thereof or at least one thickening agent, wax, coloring matter, antioxidant, perfume, essential oil, preservative, cosmetic active agent, moisturizer, vitamin, ceramide, sunscreen, surfactant, spreading agent, wetting agent, dispersing agent, antifoam, neutralizer, stabilizer, polymer and in particular fat-soluble film-forming polymer, and mixtures thereof.

17. The composition according to claim 1, provided in the form of a care and/or- or make-up product for the skin of the body or of the face, the lips, the eyelashes, the eyebrows, the hair, the scalp or the nails; an anti-sun or a self-tanning product; a hair product, in particular for dyeing, conditioning and/or or caring for the hair.

18. A method for the cosmetic treatment of keratin materials, comprising applying to the skin of the body or of the face, the lips, the nails, the hair or the eyelashes cosmetic composition as defined in claim 1.

19. A cosemetic method for caring for or making up the lips, comprising the application of a cosmetic composition as defined in claim 1.

20. A polycondensate obtained by a process comprising reacting:
from 10 to 30% by weight, relative to the total weight of the polycondensate, of one or more polyols comprising 3 to 6 hydroxyl groups;
from 30 to 80% by weight, relative to the total weight of the polycondensate, of one or more linear, branched or cyclic, saturated or unsaturated, non-aromatic monocarboxylic acids comprising 6 to 32 carbon atoms;
from 1 to 40% by weight, relative to the total weight of the polycondensate, of one or more polycarboxylic acids or cyclic anhydrides of such a polycarboxylic acid or lactones comprising at least one COOH group; and
from 0.1 to 15% by weight, relative to the total weight of the polycondensate, of one or more silicones having a hydroxyl or carboxyl functional group.

21. The composition of claim 1, further comprising 0.1 to 15% by weight, relative to the total weight of the polycondensate, of at least one silicone having a hydroxyl or carboxyl functional group, or mixtures thereof.

22. The composition according to claim 1, wherein the polyol is at least one selected from the group consisting of 1,2,4-butanetriol, 1,2,6-hexane-triol, trimethylolethane, trimethylolpropane, glycerol, pentaerythritol, erythritol, diglycerol, ditrimethylolpropane, xylitol, sorbitol, mannitol, dipentaerythritol, and triglycerol.

23. The composition according to claim 1, wherein the non-aromatic monocarboxylic acid is at least one selected from the group of unsaturated but non-aromatic monocarboxylic acids consisting of caproleic acid, obtusilic acid, undecylenic acid, dodecylenic acid, linderic acid, myristoleic acid, physeteric acid, tsuzuic acid, palmitoleic acid, oleic acid, petroselinic acid, vaccenic acid, elaidic acid, gondoic acid, gadoleic acid, erucic acid, cetoleic acid, nervonic acid, linoleic acid, linolenic acid, and arachidonic acid.

24. The composition of claim 7, wherein the cyclic anhydride is selected from the group consisting of 1,4,5,8-naphthalenetetracarboxylic acid 1,4-monoanhydride, itaconic anhydride, phthalic anhydride, trimellitic anhydride, maleic anhydride and succinic anhydride.
